(19) United States
(12) Patent Application Publication
(10) Pub. No.: US 2020/0222555 A1
(43) Pub. Date: Jul. 16, 2020

(54) ITDC COMPOSITIONS AND METHODS FOR USE IN THERAPEUTIC METHODS

(71) Applicant: Falcon Therapeutics, Inc., Durham, NC (US)

(72) Inventor: Susan B. NICHOLS, Durham, NC (US)

(21) Appl. No.: 16/744,035

(22) Filed: Jan. 15, 2020

Related U.S. Application Data
(60) Provisional application No. 62/792,837, filed on Jan. 15, 2019.

(51) Int. Cl. A61K 47/69; A61K 47/64; A61K 38/45; A61K 31/522; A61P 35/04; A61P 31/00

(52) U.S. Cl. CPC A61K 47/6901; A61K 47/6435; A61P 31/00; A61K 31/522; A61P 35/04; A61K 38/45

(57) ABSTRACT

Provided herein are methods of treating cancer by performing first and second administrations of induced tumor homing therapeutic cells to a subject. In some embodiments, the first administration comprises administering the induced tumor homing therapeutic cells to a surgical site and the second administration comprises administering the induced tumor homing therapeutic cells to a subject parentally. Also described herein are pharmaceutical compositions comprising induced tumor homing therapeutic cells.

### CROSS-REFERENCE

This application claims the benefit of U.S. Provisional Application No. 62/792,837, filed Jan. 15, 2019, which is incorporated herein by reference in its entirety.

### SUMMARY OF THE DISCLOSURE

Disclosed herein, in certain embodiments, are methods of treating a cancer, comprising: (a) performing a first administration, the first administration comprising administering a first induced tumor-homing drug carrier cell (iTDC) to a surgical space of a subject with cancer, the first iTDC comprising (i) a first recombinant nucleotide encoding a nucleic acid sequence encoding Sox2 and (ii) a first therapeutic nucleic acid sequence encoding a therapeutic payload; and (b) performing a second administration simultaneously, on the same day as the first administration, or at most about 10 days after performing the first administration, the second administration comprising parenterally administering a second iTDC to the subject, the second iTDC comprising (i) a second recombinant polynucleotide comprising a nucleic acid sequence encoding Sox2 and (ii) a second therapeutic nucleic acid sequence encoding a therapeutic payload; whereby the first and/or second iTDC expresses the therapeutic payload at a cancer cell and treats the cancer. In some embodiments, the second administration is performed simultaneously or on the same day as the first administration. In some embodiments, the second administration is performed at most about 6, 5, 4, 3, 2, or 1 day after the first administration. In some embodiments, the first iTDC is attached to or encapsulated in a scaffold. In some embodiments, the scaffold is a hydrogel. In some embodiments, the scaffold comprises gelatin and/or thrombin. In some embodiments, the gelatin is bovine-derived gelatin. In some embodiments, the thrombin is human-derived. In some embodiments, the scaffold comprises fibrin. In some embodiments, the cancer is brain cancer. In some embodiments, the brain cancer is glioblastoma (GBM). In some embodiments, the cancer is ovarian cancer. In some embodiments, the cancer is pulmonary cancer. In some embodiments, the cancer is breast cancer. In some embodiments, the breast cancer is triple negative breast cancer. In some embodiments, the cancer is melanoma. In some embodiments, the cancer is leukemia. In some embodiments, the cancer is lymphoma. In some embodiments, the first therapeutic nucleic acid sequence and/or the second therapeutic nucleic acid sequence comprise a nucleic acid sequence encoding an anti-cancer polypeptide. In some embodiments, the anti-cancer polypeptide is thymidine kinase (TK). In some embodiments, the anti-cancer polypeptide is a cytokine. In some embodiments, the cytokine is a protein, peptide, glycoprotein, chemokine, interleukin, tumor necrosis factor (TNF), monocyte chemoattractant protein (MCP), IL-1-like cytokine, gamma chain cytokine, beta chain cytokine, IL-6-like cytokine, IL-10-like cytokine, interferon, tumor necrosis factor, TGF-beta, macrophage inflammatory protein (MIP), tumor growth factor (TGF), matrix metalloprotease (MMP), or any combination thereof wherein the first therapeutic nucleic acid sequence and/or the second therapeutic nucleic acid sequence comprise a nucleic acid sequence encoding an anti-cancer therapeutic. In some embodiments, the anti-cancer therapeutic is TNF-related apoptosis-inducing ligand (TRAIL). In some embodiments, the method further comprises administering ganciclovir (GCV) or valganciclovir to the subject. In some embodiments, the GCV or valganciclovir administration is an oral administration. In some embodiments, the GCV or valganciclovir administration is performed simultaneously or on the same day as the first administration and the second administration. In some embodiments, the GCV or valganciclovir administration is performed at least about 3 days after the first administration and the second administration. In some embodiments, the method further comprises administering to the subject a chemotherapy, radiation therapy, bone marrow transplant, immunotherapy, hormone therapy, cryoablation, radiofrequency ablation, or any combination thereof. In some embodiments, administering a chemotherapy comprises administering a chemotherapeutic agent. In some embodiments, the chemotherapeutic agent is an alkylating agent, an anthracycline, a cytoskeletal disruptor, an epothilone, a histone deacetylase inhibitor, a topoisomerase I inhibitor, a topoisomerase II inhibitor, a kinase inhibitor, a nucleotide analog, a precursor analog, a peptide antibiotic, a platinum-based agent, a retinoid, a vinca alkaloid, or a combination thereof. In some embodiments, the chemotherapeutic agent is actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, or a combination thereof. In some embodiments, the first iTDC persists in the subject for at least about 3 months after the first administration. In some embodiments, the second iTDC persists in the subject for at least about 12 hours after the second administration. In some embodiments, the first iTDC and the second iTDC are autologous cells. In some embodiments, the second administration is an intracerebroventricular (ICV) injection. In some embodiments, the second administration is an intrathecal injection. In some embodiments, the second administration is an intravenous injection. In some embodiments, the second administration is an intraperitoneal injection. In some embodiments, the first iTDC and the second iTDC express CXCR4. In some embodiments, the first iTDC and the second iTDC comprise a label. In some embodiments, the label is a fluorescent label. In some embodiments, the first recombinant polynucleotide and the second recombinant polynucleotide encode a nucleic acid sequence encoding the fluorescent label. In some embodiments, the label is a magnetic resonance imaging (MRI) contrast agent or a positron emission tomography (PET) contrast agent. In some embodiments, the surgical space is a tumor resection space.

Disclosed herein, in certain embodiments, are methods of treating a cancer comprising: performing a first administration, the first administration comprising administering a first induced tumor-homing drug carrier cell (iTDC) to a surgical space of a subject with cancer, a surface of the surgical space comprising a matrix material, the first iTDC comprising (i) a first recombinant nucleotide encoding a nucleic acid sequence encoding Sox2 and (ii) a first therapeutic nucleic acid sequence encoding a therapeutic payload; and performing a second administration simultaneously, on the same day as the first administration, or at most about 10 days after performing the first administration, the second administration comprising parenterally administering a second iTDC to the subject, the second iTDC expressing a therapeutic payload and comprising (i) a second recombinant polynucleotide comprising a nucleic acid sequence encoding Sox2 and (ii) a second therapeutic nucleic acid sequence encoding a therapeutic payload, whereby the first and/or second iTDC expresses the therapeutic payload at a cancer cell and treats the cancer. In some embodiments, the second administration is performed simultaneously or on the same day as the first administration. In some embodiments, the second administration is performed at most about 7, 6, 5, 4, 3, 2, or 1 day after the first administration. In some embodiments, the matrix material comprises a hemostatic composition. In some embodiments, the treated surgical space is free of an excess of the hemostatic composition. In some embodiments, the matrix material is flowable. In some embodiments, the first iTDC is administered to the matrix material. In some embodiments, the matrix material is a hydrogel. In some embodiments, the matrix material comprises gelatin and/or thrombin. In some embodiments, the gelatin is bovine-derived gelatin. In some embodiments, the thrombin is human-derived. In some embodiments, the matrix material comprises fibrin. In some embodiments, the cancer is brain cancer. In some embodiments, the brain cancer is glioblastoma (GBM). In some embodiments, the cancer is ovarian cancer. In some embodiments, the cancer is pulmonary cancer. In some embodiments, the cancer is breast cancer. In some embodiments, the breast cancer is triple negative breast cancer. In some embodiments, the cancer is melanoma. In some embodiments, the cancer is leukemia. In some embodiments, the cancer is lymphoma. In some embodiments, the cancer is pancreatic cancer. In some embodiments, the first therapeutic nucleic acid sequence and/or the second therapeutic nucleic acid sequence comprise a nucleic acid sequence encoding an anti-cancer polypeptide. In some embodiments, the anti-cancer polypeptide is thymidine kinase (TK). In some embodiments, the anti-cancer polypeptide is a cytokine. In some embodiments, the cytokine is a protein, peptide, glycoprotein, chemokine, interleukin, tumor necrosis factor (TNF), monocyte chemoattractant protein (MCP), IL-1-like cytokine, gamma chain cytokine, beta chain cytokine, IL-6-like cytokine, IL-10-like cytokine, interferon, tumor necrosis factor, TGF-beta, macrophage inflammatory protein (MIP), tumor growth factor (TGF), matrix metalloprotease (MMP), or any combination thereof. In some embodiments, wherein the first therapeutic nucleic acid sequence and/or the second therapeutic nucleic acid sequence comprise a nucleic acid sequence encoding an anti-cancer therapeutic. In some embodiments, the anti-cancer therapeutic is TNF-related apoptosis-inducing ligand (TRAIL). In some embodiments, the method further comprises administering ganciclovir (GCV) or valganciclovir to the subject. In some embodiments, the GCV or valganciclovir administration is an oral administration. In some embodiments, the GCV or valganciclovir administration is performed simultaneously or on the same day as the first administration and the second administration. In some embodiments, the GCV or valganciclovir administration is performed at least about 3 days after the first administration and the second administration. In some embodiments, the method further comprises administering to the subject a chemotherapy, radiation therapy, bone marrow transplant, immunotherapy, hormone therapy, cryoablation, radiofrequency ablation, or any combination thereof. In some embodiments, administering a chemotherapy comprises administering a chemotherapeutic agent. In some embodiments, the chemotherapeutic agent is an alkylating agent, an anthracycline, a cytoskeletal disruptor, an epothilone, a histone deacetylase inhibitor, a topoisomerase I inhibitor, a topoisomerase II inhibitor, a kinase inhibitor, a nucleotide analog, a precursor analog, a peptide antibiotic, a platinum-based agent, a retinoid, a vinca alkaloid, or a combination thereof. In some embodiments, the chemotherapeutic agent is actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, or a combination thereof. In some embodiments, the first iTDC persists in the subject for at least about 3 months after the first administration. In some embodiments, the second iTDC persists in the subject for at least about 12 hours after the second administration. In some embodiments, the first iTDC and the second iTDC are autologous cells. In some embodiments, the second administration is an intracerebroventricular (ICV) injection. In some embodiments, the second administration is an intrathecal injection. In some embodiments, the second administration is an intravenous injection. In some embodiments, the second administration is an intraperitoneal injection. In some embodiments, the first iTDC and the second iTDC express CXCR4. In some embodiments, the first iTDC and the second iTDC comprise a label. In some embodiments, the label is a fluorescent label. In some embodiments, the first recombinant polynucleotide and the second recombinant polynucleotide encode a nucleic acid sequence encoding the fluorescent label. In some embodiments, the label is a magnetic resonance imaging (MRI) contrast agent or a positron emission tomography (PET) contrast agent. In some embodiments, the treated surgical space is a treated tumor resection space.

### DETAILED DESCRIPTION OF THE DISCLOSURE

While preferred embodiments of the subject matter disclosed herein have been shown and described herein, it will be obvious to those skilled in the art that such embodiments are provided by way of example only. Numerous variations, changes, and substitutions will now occur to those skilled in the art without departing from the subject matter disclosed herein. It should be understood that various alternatives to the embodiments of the subject matter disclosed herein may be employed in practicing the subject matter disclosed herein. It is intended that the following claims define the scope of the subject matter disclosed herein and that methods and structures within the scope of these claims and their equivalents be covered thereby.

### I. Exemplary Embodiments

A. Methods of Treating Diseases

Tumor resection—surgical removal of all or part of a cancerous tumor—is a common treatment for cancer patients. In some instances, cancer reoccurs after tumor resection. In some instances, metastatic or surgery-induced migration of tumor cells away from the tumor site precludes surgical resection from being effective. Even with the best imaging methods available, it is difficult to detect cancerous cells that have migrated away from a primary tumor. Glioblastomas, for example, are characterized by rapid and highly infiltrative tumor cell migration and invasion. As a result, Glioblastomas are often difficult, if not impossible, to treat by surgical resection.

Provided herein are methods of treating a cancer by administering induced tumor-homing drug carrier cells (iTDCs) both directly to a surgical site following surgical resection, and systemically to a target tissue (e.g., by intracerebroventricular (ICV) injection, intrathecal injection, intravenous injection, intraperitoneal injection, etc.). In some embodiments, the iTDCs preferentially accumulate at (e.g., home to, migrate to) a tumor. In some embodiments, the iTDCs are loaded with a therapeutic agent for treating the cancer. In some embodiments, the methods described herein allow for the targeting and treating both cancer cells in or near the surgical resection site and cancer cells that have migrated away from the resection site (and are therefore difficult to detect and/or in an area where resection is not feasible). In some embodiments, the cancer is a solid cancer. In some embodiments, the cancer is a lung cancer, breast cancer (e.g., triple negative breast cancer), brain cancer (e.g., glioblastoma), ovarian cancer, pulmonary cancer, melanoma, leukemia, lymphoma, and pancreatic cancer.

Disclosed herein in certain embodiments, are methods of treating a cancer, comprising: (a) performing a first administration, the first administration comprising administering a first induced tumor-homing drug carrier cell (iTDC) to a surgical space of a subject with cancer, the first iTDC comprising (i) a first recombinant nucleotide encoding a nucleic acid sequence encoding Sox2 and (ii) a first therapeutic nucleic acid sequence encoding a therapeutic payload; and (b) performing a second administration simultaneously, on the same day as the first administration, or at most about 10 days after performing the first administration, the second administration comprising parenterally administering a second iTDC to the subject, the second iTDC comprising (i) a second recombinant polynucleotide comprising a nucleic acid sequence encoding Sox2 and (ii) a second therapeutic nucleic acid sequence encoding a therapeutic payload; whereby the first and/or second iTDC expresses the therapeutic payload at a cancer cell and treats the cancer.

i. Induced Tumor Homing Drug Carrier Cells

In some embodiments, the first and second therapeutic cells are induced tumor-homing drug carrier cells (iTDC). The iTDCs described herein are made by any suitable method. In some embodiments, the iTDCs are transdifferentiated ex vivo before administration to the individual. In some embodiments, the iTDCs are autologous. In some embodiments, the iTDCs are allogenic. In some embodiments, the iTDCs are fresh, i.e., not frozen or previously frozen. In some embodiments, the iTDCs are cryopreserved (frozen). In some embodiments, the iTDCs are frozen and stored for later use (for example to facilitate transport). In some embodiments, the frozen iTDCs are administered to the individual after being thawed.

In some embodiments, the iTDCs are produced by transfecting a somatic cell with an exogenous nucleic acid sequence encoding a transdifferentiation factor and culturing the transfected somatic cell in a progenitor medium, thereby transforming the somatic cell into an induced tumor-homing drug carrier cell. In some embodiments, the iTDC is not a pluripotent stem cell or an induced neural stem cell. In some embodiments, the first and second iTDCs are transdifferentiated from a somatic cell autologous to the individual. In some embodiments, the first and second iTDCs are non-proliferative. In some embodiments, the first and second iTDCs comprise a first recombinant polynucleotide comprising a nucleic acid sequence encoding Sox2. In some embodiments, iTDCs cells are somatic cells (e.g., differentiated cells) that were induced into a lineage different that their original lineage. In some embodiments, the somatic cells are not embryonic stem cells. In some embodiments, iTDCs are adult cells (e.g., somatic cells that can differentiate into limited cell types) that that were induced into a lineage different that their original lineage.

In some embodiments, the somatic cell or primary cell is any cell from the body of a subject other than gametocyte, germ cell, or undifferentiated stem cell. In some instances, the somatic cell or primary cell comprises a fibroblast cell, a muscle cell, an epithelial cell, or a nerve cell. Epithelial cells include squamous cells, cuboidal cells, and/or columnar cells. Cells of the muscle comprise skeletal muscle, smooth muscle, and/or cardiac muscle.

In some embodiments, the somatic cells or adult cells are fibroblasts. Fibroblast cells synthesize extracellular matrix and collagen and are the most common type of cells within the connective tissue. In some instances, the fibroblast cells are skin fibroblast cells. Skin cells can be collected from a skin punch as a stand-alone procedure or from a surgical incision, e.g., during an accompanying surgery procedure; and can be collected from any area, including, but not limited to, arm (e.g, forearm), leg, or scalp. In some embodiments, the somatic cell or primary cell utilized with a method described herein is a fibroblast cell (e.g., a skin fibroblast cell). In some embodiments, a recombinant polynucleotide described herein is introduced into the fibroblast cell (e.g., the skin fibroblast cell), wherein the recombinant polynucleotide gives rise, upon transcription, to a factor that contributes to the reprogramming of the fibroblast cell (e.g., the skin fibroblast cell) into a therapeutic or engineered stem cell.

In some embodiments, the human somatic or human adult cells used to generate the therapeutic cells include fibroblasts, keratinocytes, peripheral blood cells, renal epithelial cells, monocytes, adipose cells, or hepatocytes. In some embodiments, any cells other than germ cells of mammalian origin (e.g., humans, mice, monkeys, pigs, rats, etc.) are used as starting material for the production of the therapeutic cells. Examples include, but are not limited to, keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), contractile cells (e.g., smooth muscle cells), cells of the blood and the immune system (e.g., T lymphocytes), sense-related cells (e.g., rod cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells), and the like. There is no limitation on the degree of cell differentiation and/or the age of the animal from which cells are collected. In some embodiments, undifferentiated progenitor cells (including somatic stem cells) and finally differentiated mature cells are used alike as sources of somatic cells and adult cells in the present disclosure. Non-limiting examples of undifferentiated progenitor cells include tissue stem cells (somatic stem cells) such as neural stem cells, hematopoietic stem cells, mesenchymal stem cells, and dental pulp stem cells.

In some instances, the somatic cell or primary cell is an autologous cell obtained from the subject upon which treatment is to be administered. In other instances, the somatic cell or primary cell is an allogenic cell obtained from a subject upon which the subject will not receive treatment. In some embodiments, this subject is a healthy subject.

In some embodiments, the first and second iTDCs are transdifferentiated by administration of a recombinant polynucleotide. In some embodiments, the recombinant polynucleotide comprises Sox2. In some embodiments, the recombinant polynucleotide comprises one or more factors from the group consisting of: Sox2, Oct3/4, c-Myc, Klf4, Nanog, Lin28, Ascl1, Brn2, Myt11, Olig2, and Zicl. In some embodiments, the recombinant polynucleotide is a vector. In some embodiments, the vector is a viral vector. In some embodiments, the vector is a lentiviral vector, an adenoviral vector, an adeno-associated virus (AAV), or a retroviral vector.

In some embodiments, a plurality of somatic or adult cells is virally co-transduced with a Sox2 gene in order to produce a plurality of induced therapeutic cells. In some embodiments, a plurality of somatic or adult cells is virally co-transduced with Oct3/4, Sox2, c-Myc, Klf4, Nanog genes, or any combination thereof, in order to produce a plurality of induced therapeutic cells. In some embodiments, viral co-transduction with Oct3/4, c-Myc, Klf4, Nanog, Lin28, Ascl1, Brn2, Myt11, Olig2, and Zicl is not necessary to produce the therapeutic cells.

In some embodiments, the iTDCs are cultured in a progenitor medium, such as a neural progenitor medium. Feeder cells, as known in the art, are additional cells grown in the same culture dish or container, often as a layer (e.g., a mouse fibroblast layer on the culture dish) to support cell growth. “Progenitor medium”, as used herein, is a medium or media, for example, incorporating supplements, small molecule inhibitors, and growth factors, that promotes the transdifferentiation (TD) of somatic cells into neural stem cells. In some embodiments, the progenitor medium includes one or more ingredients selected from: a cell culture medium containing growth-promoting factors and/or a nutrient mixture (e.g., DMEM/F12, MEM/D-valine, neurobasal medium etc., including mixtures thereof); media supplements containing hormones, proteins, vitamins and/or amino acids (e.g., N2 supplement, B27 supplement, non-essential amino acids (NEAA), L-glutamine, GlutaMAX™, BSA, insulin, all trans retinoic acid, etc. including mixtures thereof); and optionally small molecule inhibitors (e.g., SB431542 (BMP inhibitor), LDN193189 (TGF-f31 inhibitor), CHIR99021 (GSK3f3 inhibitor), etc., including mixtures thereof). In some embodiments, ingredients also include one or more of beta-mercaptoethanol, transferrin; sodium selenite; and cAMP. In some embodiments, suitable concentrations of each of these ingredients are known to those of skill in the art and/or are empirically determined. Example concentrations of ingredients is also provided in Example 25 below. In some embodiments, the progenitor medium is a premade medium, such as STEMdiff™ Neural Induction Medium (STEMCELL™ Technologies, Vancouver, British Columbia, Canada).

In some instances, the therapeutic cell described herein is characterized with one or more biomarkers, such as for example, nestin, glial fibrillary acidic protein (or GFAP), Tuj-1 (neuron-specific class III beta-tubulin or βIII tubulin), Nanog, or OCT3/4.

In some instances, the therapeutic cell is further characterized with the expression of CXCR4 (e.g., the therapeutic cell is CXCR4 positive).

ii. Anti-Cancer Polypeptides and Therapeutics

In some embodiments, the first and second iTDCs are transdifferentiated by administration of a recombinant polynucleotide. In some embodiments, the recombinant polynucleotide comprises Sox2. In some embodiments, the recombinant polynucleotide comprises one or more factors from the group consisting of: Sox2, Oct3/4, c-Myc, Klf4, Nanog, Lin28, Ascl1, Brn2, Myt11, Olig2, and Zicl. In some embodiments, the recombinant polynucleotide is a vector. In some embodiments, the vector is a viral vector. In some embodiments, the vector is a lentiviral vector, an adenoviral vector, an adeno-associated virus (AAV), or a retroviral vector.

In some embodiments, a plurality of somatic or adult cells is virally co-transduced with a Sox2 gene in order to produce a plurality of induced therapeutic cells. In some embodiments, a plurality of somatic or adult cells is virally co-transduced with Oct3/4, Sox2, c-Myc, Klf4, Nanog genes, or any combination thereof, in order to produce a plurality of induced therapeutic cells. In some embodiments, viral co-transduction with Oct3/4, c-Myc, Klf4, Nanog, Lin28, Ascl1, Brn2, Myt11, Olig2, and Zicl is not necessary to produce the therapeutic cells.

In some embodiments, the first and second iTDCs further comprises a therapeutic nucleic acid sequence encoding an anti-cancer polypeptide. In some embodiments, the first recombinant polypeptide and/or the second recombinant polypeptide comprise the therapeutic nucleic acid sequence encoding an anti-cancer polypeptide. In some embodiments, the first recombinant polypeptide and/or the second recombinant polypeptide comprise the therapeutic nucleic acid sequence encoding one or more anti-cancer polypeptides. In some embodiments, the anti-cancer polypeptide is used to target the therapeutic cell to a tumor cell. In some embodiments, the therapeutic cell delivers an anti-cancer polypeptide to a tumor cell, thereby treating and/or delaying the progression of cancer. In some embodiments, the anti-cancer polypeptide is a payload delivered to a tumor cell by the therapeutic cell.

In some embodiments, the anti-cancer polypeptide is thymidine kinase (TK). In some embodiments, the anti-cancer polypeptide is a herpes simplex virus (HSV) TK. In some embodiments, the first and second iTDCs express TK. In some embodiments, the first and second iTDCs secrete TK. In some embodiments, the therapeutic cell delivers TK to a tumor cell, which treats and/or delays the progression of cancer. In some embodiments, TK is a payload delivered to a tumor cell by the therapeutic cell.

In some embodiments, the methods disclosed herein comprise administering ganciclovir (GCV) or valganciclovir to the subject. Systemic administration of GCV induces selective apoptosis in cells transduced with the TK gene. TK is able to phosphorylate the GCV, turning it into monophosphorylated GCV, which is subsequently tri-phosphorylated by cellular kinases. This product blocks the DNA replication, causing its fragmentation and apoptosis. In some embodiments, thymidine kinase enzymes phosphorylate the thymidine to thymidine triphosphate, for further integration into DNA. The HSV thymidine kinase, unlike to human thymidine kinase, is able to phosphorylate ganciclovir to convert it to GCV-P, once this occurs the cellular kinases phosphorylate it for later integration into the DNA, which leads to the arrest of its synthesis and therefore, the cell death. Thus, administration of GCV to the subject leads to apoptosis of tumor cells targeted by the iTDCs of the disclosure.

In some embodiments, the GCV or valganciclovir administration is an oral administration. In some embodiments, the GCV or valganciclovir administration is intravenous, intratumoral, intradermal, subcutaneous (S.C., s.q., sub-Q, Hypo), intramuscular, intra-arterial, intramedullary, intracardiac, intra-articular (joint), intrasynovial (joint fluid area), intracranial, intraspinal, intrathecal (spinal fluids), intraduodenal, intramedullary, intraosseous, intrathecal, intravascular, intravitreal, epidural administration, or any combinations thereof.

In some embodiments, ganciclovir is administered to the subject in an oral dose of about 500 milligrams (mg). In some embodiments, ganciclovir is administered to the subject in an oral dose of about 1000 mg. In some embodiments, ganciclovir is administered to the subject in an oral dose of about 250 mg to about 2,000 mg. In some embodiments, ganciclovir is administered to the subject in an oral dose of about 250 mg to about 500 mg, about 250 mg to about 600 mg, about 250 mg to about 700 mg, about 250 mg to about 800 mg, about 250 mg to about 900 mg, about 250 mg to about 1,000 mg, about 250 mg to about 1,500 mg, about 250 mg to about 2,000 mg, about 500 mg to about 600 mg, about 500 mg to about 700 mg, about 500 mg to about 800 mg, about 500 mg to about 900 mg, about 500 mg to about 1,000 mg, about 500 mg to about 1,500 mg, about 500 mg to about 2,000 mg, about 600 mg to about 700 mg, about 600 mg to about 800 mg, about 600 mg to about 900 mg, about 600 mg to about 1,000 mg, about 600 mg to about 1,500 mg, about 600 mg to about 2,000 mg, about 700 mg to about 800 mg, about 700 mg to about 900 mg, about 700 mg to about 1,000 mg, about 700 mg to about 1,500 mg, about 700 mg to about 2,000 mg, about 800 mg to about 900 mg, about 800 mg to about 1,000 mg, about 800 mg to about 1,500 mg, about 800 mg to about 2,000 mg, about 900 mg to about 1,000 mg, about 900 mg to about 1,500 mg, about 900 mg to about 2,000 mg, about 1,000 mg to about 1,500 mg, about 1,000 mg to about 2,000 mg, or about 1,500 mg to about 2,000 mg. In some embodiments, ganciclovir is administered to the subject in an oral dose of about 250 mg, about 500 mg, about 600 mg, about 700 mg, about 800 mg, about 900 mg, about 1,000 mg, about 1,500 mg, or about 2,000 mg. In some embodiments, ganciclovir is administered to the subject in an oral dose of at least about 250 mg, about 500 mg, about 600 mg, about 700 mg, about 800 mg, about 900 mg, about 1,000 mg, or about 1,500 mg. In some embodiments, ganciclovir is administered to the subject in an oral dose of at most about 500 mg, about 600 mg, about 700 mg, about 800 mg, about 900 mg, about 1,000 mg, about 1,500 mg, or about 2,000 mg.

In some embodiments, ganciclovir is administered to the subject in an intravenous dose of about 5 milligrams/kilogram (mg/kg). In some embodiments, ganciclovir is administered to the subject in an intravenous dose of about 0.5 mg/kg to about 15 mg/kg. In some embodiments, ganciclovir is administered to the subject in an intravenous dose of about 0.5 mg/kg to about 1 mg/kg, about 0.5 mg/kg to about 1.5 mg/kg, about 0.5 mg/kg to about 2 mg/kg, about 0.5 mg/kg to about 2.5 mg/kg, about 0.5 mg/kg to about 3 mg/kg, about 0.5 mg/kg to about 3.5 mg/kg, about 0.5 mg/kg to about 4 mg/kg, about 0.5 mg/kg to about 5 mg/kg, about 0.5 mg/kg to about 6 mg/kg, about 0.5 mg/kg to about 10 mg/kg, about 0.5 mg/kg to about 15 mg/kg, about 1 mg/kg to about 1.5 mg/kg, about 1 mg/kg to about 2 mg/kg, about 1 mg/kg to about 2.5 mg/kg, about 1 mg/kg to about 3 mg/kg, about 1 mg/kg to about 3.5 mg/kg, about 1 mg/kg to about 4 mg/kg, about 1 mg/kg to about 5 mg/kg, about 1 mg/kg to about 6 mg/kg, about 1 mg/kg to about 10 mg/kg, about 1 mg/kg to about 15 mg/kg, about 1.5 mg/kg to about 2 mg/kg, about 1.5 mg/kg to about 2.5 mg/kg, about 1.5 mg/kg to about 3 mg/kg, about 1.5 mg/kg to about 3.5 mg/kg, about 1.5 mg/kg to about 4 mg/kg, about 1.5 mg/kg to about 5 mg/kg, about 1.5 mg/kg to about 6 mg/kg, about 1.5 mg/kg to about 10 mg/kg, about 1.5 mg/kg to about 15 mg/kg, about 2 mg/kg to about 2.5 mg/kg, about 2 mg/kg to about 3 mg/kg, about 2 mg/kg to about 3.5 mg/kg, about 2 mg/kg to about 4 mg/kg, about 2 mg/kg to about 5 mg/kg, about 2 mg/kg to about 6 mg/kg, about 2 mg/kg to about 10 mg/kg, about 2 mg/kg to about 15 mg/kg, about 2.5 mg/kg to about 3 mg/kg, about 2.5 mg/kg to about 3.5 mg/kg, about 2.5 mg/kg to about 4 mg/kg, about 2.5 mg/kg to about 5 mg/kg, about 2.5 mg/kg to about 6 mg/kg, about 2.5 mg/kg to about 10 mg/kg, about 2.5 mg/kg to about 15 mg/kg, about 3 mg/kg to about 3.5 mg/kg, about 3 mg/kg to about 4 mg/kg, about 3 mg/kg to about 5 mg/kg, about 3 mg/kg to about 6 mg/kg, about 3 mg/kg to about 10 mg/kg, about 3 mg/kg to about 15 mg/kg, about 3.5 mg/kg to about 4 mg/kg, about 3.5 mg/kg to about 5 mg/kg, about 3.5 mg/kg to about 6 mg/kg, about 3.5 mg/kg to about 10 mg/kg, about 3.5 mg/kg to about 15 mg/kg, about 4 mg/kg to about 5 mg/kg, about 4 mg/kg to about 6 mg/kg, about 4 mg/kg to about 10 mg/kg, about 4 mg/kg to about 15 mg/kg, about 5 mg/kg to about 6 mg/kg, about 5 mg/kg to about 10 mg/kg, about 5 mg/kg to about 15 mg/kg, about 6 mg/kg to about 10 mg/kg, about 6 mg/kg to about 15 mg/kg, or about 10 mg/kg to about 15 mg/kg. In some embodiments, ganciclovir is administered to the subject in an intravenous dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, about 6 mg/kg, about 10 mg/kg, or about 15 mg/kg. In some embodiments, ganciclovir is administered to the subject in an intravenous dose of at least about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, about 6 mg/kg, or about 10 mg/kg. In some embodiments, ganciclovir is administered to the subject in an intravenous dose of at most about 1 mg/kg, about 1.5 mg/kg, about 2 mg/kg, about 2.5 mg/kg, about 3 mg/kg, about 3.5 mg/kg, about 4 mg/kg, about 5 mg/kg, about 6 mg/kg, about 10 mg/kg, or about 15 mg/kg.

In some embodiments, the GCV or valganciclovir administration is performed simultaneously or on the same day as the first administration and the second administration. In some embodiments, the GCV or valganciclovir administration is performed at least about 3 days after the first administration and the second administration. In some embodiments, the GCV or valganciclovir administration is performed at least about 2 days after the first administration and the second administration. In some embodiments, the GCV or valganciclovir administration is performed at least about 1 day after the first administration and the second administration. In some embodiments, the GCV or valganciclovir administration is performed about 0.1 days to about 5 days after the first administration and the second administration. In some embodiments, the GCV or valganciclovir administration is performed about 0.1 days to about 0.5 days, about 0.1 days to about 1 day, about 0.1 days to about 2 days, about 0.1 days to about 3 days, about 0.1 days to about 4 days, about 0.1 days to about 5 days, about 0.5 days to about 1 day, about 0.5 days to about 2 days, about 0.5 days to about 3 days, about 0.5 days to about 4 days, about 0.5 days to about 5 days, about 1 day to about 2 days, about 1 day to about 3 days, about 1 day to about 4 days, about 1 day to about 5 days, about 2 days to about 3 days, about 2 days to about 4 days, about 2 days to about 5 days, about 3 days to about 4 days, about 3 days to about 5 days, or about 4 days to about 5 days after the first administration and the second administration. In some embodiments, the GCV or valganciclovir administration is performed about 0.1 days, about 0.5 days, about 1 day, about 2 days, about 3 days, about 4 days, or about 5 days after the first administration and the second administration. In some embodiments, the GCV or valganciclovir administration is performed at least about 0.1 days, about 0.5 days, about 1 day, about 2 days, about 3 days, or about 4 days after the first administration and the second administration. In some embodiments, the GCV or valganciclovir administration is performed at most about 0.5 days, about 1 day, about 2 days, about 3 days, about 4 days, or about 5 days after the first administration and the second administration.

In some embodiments, the first and/or second iTDC comprises a nucleic acid sequence encoding an anti-cancer therapeutic. In some embodiments, the first recombinant polypeptide and/or the second recombinant polypeptide comprise a nucleic acid sequence encoding one or more anti-cancer therapeutics. In some embodiments, the anti-cancer therapeutic is used to target the therapeutic cell to a tumor cell. In some embodiments, the iTDC delivers an anti-cancer therapeutic to a tumor cell, thereby treating and/or delaying the progression of cancer. In some embodiments, the anti-cancer therapeutic is a payload delivered to a tumor cell by the iTDC.

In some embodiments, the anti-cancer therapeutic is TNF-related apoptosis-inducing ligand (TRAIL) or secretable tumor necrosis factor (TNF)-related apoptosis-inducing ligand (S-TRAIL). In some embodiments, the first and second iTDCs express TRAIL. In some embodiments, the first and second iTDCs secrete TRAIL. In some embodiments, the iTDC delivers TRAIL to a tumor cell, thereby treating and/or delaying the progression of cancer. In some embodiments, the TRAIL is a payload delivered to a tumor cell by the iTDC. In some embodiments, the iTDC secretes TRAIL. In some embodiments, the iTDC homes to a tumor site and secretes TRAIL near the vicinity of a tumor. In some embodiments, TRAIL binds to the tumor cell and induces apoptosis.

In some embodiments, the anti-cancer polypeptide is a cytokine. In some embodiments, the anti-cancer therapeutic is a cytokine. In some embodiments, the cytokine is used to target the iTDC to a tumor cell. In some embodiments, the iTDC delivers a cytokine to a tumor cell, thereby treating and/or delaying the progression of cancer. In some embodiments, the cytokine is a payload delivered to a tumor cell by the iTDC.

In some embodiments, the cytokine is a protein, peptide, glycoprotein, chemokine, interleukin, tumor necrosis factor (TNF), monocyte chemoattractant protein (MCP), IL-1-like cytokine, gamma chain cytokine, beta chain cytokine, IL-6-like cytokine, IL-10-like cytokine, interferon, tumor necrosis factor, TGF-beta, macrophage inflammatory protein (MIP), tumor growth factor (TGF), matrix metalloprotease (MMP), or any combination thereof.

In some embodiments, the cytokines comprise interleukins, tumor necrosis factors (TNFs), monocyte chemoattractant proteins (MCPs), macrophage inflammatory proteins (MIPs), tumor growth factors (TGFs), and matrix metalloproteases (MMPs).

In some embodiments, cytokines are classified as proteins, peptides, glycoproteins, chemokines, interleukins, tumor necrosis factors (TNFs), monocyte chemoattractant proteins (MCPs), IL-1-like cytokines, gamma chain cytokines, beta chain cytokines, IL-6-like cytokines, IL-10-like cytokines, interferons, tumor necrosis factors, TGF-beta, macrophage inflammatory proteins (MIPs), tumor growth factors (TGFs), and matrix metalloproteases (MMPs). For example, in some embodiments, cytokines are interleukins, such as IL-1-like, IL-1α (hematopoietin-1), IL-1β (catabolin), IL-1RA (IL-1 receptor antagonist), IL-18 (interferon-γ inducing factor), Common g chain (CD132), IL-2 (T cell growth factor), IL-4 (BSF-1), IL-7, IL-9 (T cell growth factor P40), IL-13 (P600), IL-15, Common b chain (CD131), IL-3 (multipotential CSF, MCGF), IL-5 (BCDF-1), GM-CSF (CSF-2), IL-6-like, IL-6 (IFN-β2, BSF-2), IL-11 (AGIF), G-CSF (CSF-3), IL-12 (NK cell stimulatory factor), LIF (leukemia inhibitory factor), OSM (oncostatin M), IL-10-like, IL-10 (CSIF), IL-20, IL-14 (HMW-BCGF), IL-16 (LCF), and IL-17 (CTLA-8); interferons, such as IFN-α, IFN-β, and IFN-γ, tumor necrosis factors (TNFs), such as CD154 (CD40L, TRAP), LT-β, TNF-α (cachectin), TNF-β (LT-α), 4-1BBL, APRIL (TALL-2), CD70 (CD27L), CD153 (CD30L), CD178 (FasL), GITRL, LIGHT, OX40L, TALL-1, TRAIL (Apo2L), TWEAK (Apo3L), and TRANCE (OPGL); tumor growth factors, such as TGF-β1, (TGF-β), TGF-β2, and TGF-β3; and hematopoietins, such as Epo (erythropoietin), Tpo (MGDF), Flt-3L, SCF (stem cell factor, c-kit ligand), M-CSF (CSF-1), and MSP (Macrophage stimulating factor). In some embodiments, other cytokines include MST-1, CD40LG (TNFSF5), IFNA2, IL10, IL13, IL17C, IL1A, IL1B, IL1F10, IL36RN, IL36A, IL37, IL36B, IL36G, IL21, IL22, IL5, IL8, IL9, LTA, LTB, MIF, AIMP1, SPP1, and TNF. In some embodiments, exemplary, cytokine receptors are IFNA2, IL10RA, IL10RB, IL13, IL13RA1, IL5RA, IL9, and IL9R.

In some embodiments, chemokines are a family of small cytokines, or proteins secreted by cells. In some embodiments, some chemokines are pro-inflammatory and are induced during an immune response to recruit cells of an immune system to a site of infection, while others are homeostatic and are involved in controlling the migration of cells during normal processes of tissue maintenance or development. For example, in some embodiments, chemokines are XCL1 (lymphotactin a, SCM-1a, ATAC), XCL2 (lymphotactin b, SCM-1b, ATAC,) CCL1 (1-309), CCL2 (MCP-1, MCAF), CCL3 (MIP-1α, LD78α), CCL4 (MIP-1β, LAG-1, ACT-2), CCL5 (RANTES), CCL7 (MCP-3), CCL8 (MCP-2), CCL11 (eotaxin), CCL13 (MCP-4), CCL14 (HCC-1), CCL15 (HCC-2, Lkn-1, MIP-1d, MIP-5), CCL16 (HCC-4, LEC, LMC, LCC-1), CCL17 (TARC), CCL18 (DC-CK1, PARC, AMAC-a, MIP-4), CCL19 (MIP-3β, ELC, exodus-3), CCL20 (MIP-3α, LARC, exodus-1), CCL21 (6Ckine, SLC, exodus-2), CCL22 (MDC, STCP-1), CCL23 (MPIF-1, MIP-3, CKb-8), CCL24 (MPIF-2, eotaxin-2, CKb-6), CCL25 (TECK, MIP-4a), CCL26 (eotaxin-3), CCL27 (Eskine, CTACK, ILC), CXCL1 (GROa, MGSA-a), CXCL2 (GROb, MGSA-b, MIP-2a), CXCL3 (GROg, MGSA-g, MIP-2b), CXCL4 (PF4, oncostatin A), CXCL5 (ENA-78, CXCL6 (GCP-2), CXCL7 (NAP-2, PPBP), CXCL8 (IL-8, NAP-1, NAF, MDNCF), CXCL9 (Mig), CXCL10 (IP-10), CXCL11(I-TAC), CXCL12 (SDF-1α/β), CXCL13 (BLC, BCA-1), CXCL14 (BRAK), CX3CL1 (fractaline). In some embodiments, chemokine receptors are CCL13 (mcp-4), CCR1, CCR2, CCR3, CCR4, CCR5, CCR6, CCR7, CCR8, CCR9, CX3CR1, CXCR1, XCR1 (CCXCR1). In some embodiments, other proteins involved in inflammation are ABCF1, BCL6, C3, C4A, CEBPB, CRP, CARD18, IL1R1, IL1RN, CXCR2, LTB4R, and TOLLIP.

In some embodiments, the anti-cancer therapeutic and/or the anti-cancer polypeptide are antibodies. In some embodiments, the antibody is used to target the iTDC to a tumor cell. In some embodiments, the antibody is a payload delivered to a tumor cell by the iTDC. In some embodiments, the iTDC delivers an antibody to a tumor cell, thereby treating and/or delaying the progression of cancer. In some embodiments, the antibody is Adalimumab, Bezlotoxumab, Avelumab, Dupilumab, Durvalumab, Ocrelizumab, Brodalumab, Reslizumab, Olaratumab, Daratumumab, Elotuzumab, Necitumumab, Infliximab, Obiltoxaximab, Atezolizumab, Secukinumab, Mepolizumab, Nivolumab, Alirocumab, Idarucizumab, Evolocumab, Dinutuximab, Bevacizumab, Pembrolizumab, Ramucirumab, Vedolizumab, Siltuximab, Alemtuzumab, Trastuzumabemtansine, Pertuzumab, Infliximab, Obinutuzumab, Brentuximab, Raxibacumab, Belimumab, Ipilimumab, Denosumab, Denosumab, Ofatumumab, Besilesomab, Tocilizumab, Canakinumab, Golimumab, Ustekinumab, Certolizumabpegol, Catumaxomab, Eculizumab, Ranibizumab, Panitumumab, Natalizumab, Catumaxomab, Bevacizumab, Omalizumab, Cetuximab, Efalizumab, Ibritumomabtiuxetan, Fanolesomab, Adalimumab, Tositumomab and iodine 131 tositumomab, Alemtuzumab, Trastuzumab, Gemtuzumab ozogamicin, Infliximab, Palivizumab, Necitumumab, Basiliximab, Rituximab, Votumumab, Sulesomab, Arcitumomab, Imiciromab, Capromab, Nofetumomab, Abciximab, Satumomab, Muromonab-CD3, or any combination thereof.

In some embodiments, the anti-cancer therapeutic and/or the anti-cancer polypeptide are peptides. Peptides, which are short chains of amino acid monomers linked by peptide bonds, can specifically bind to tumor cells with low toxicity to normal tissues. In some embodiments, the peptide is used to target the iTDC to a tumor cell. In some embodiments, the iTDC delivers a peptide to a tumor cell, thereby treating and/or delaying the progression of cancer. In some embodiments, the peptide is a payload delivered to a tumor cell by the iTDC. In some embodiments, the peptide is a tumor-targeting peptide. In some embodiments, the peptide is a peptide targeting a signal transduction pathway, a peptide targeting the cell cycle, a peptide inducing cell death, a peptide targeting tumor suppressor proteins, a peptide targeting transcription factors, or any combination thereof. In some embodiments, the peptide is a synthetic peptide. In some embodiments, the peptide is a native peptide. In some embodiments, the peptide is RGD, NGR, JNK interacting protein, bortezomib, degarelix acetate, dactinomycin, goserelin, or any combination thereof.

In some embodiments, the peptide is an antimicrobial peptide, a cell penetrating peptide, a tumor targeting peptide, a therapeutic peptide targeting a transduction pathway, a therapeutic peptide targeting a cell cycle, a therapeutic peptide inducing cell death, a therapeutic peptide targeting a tumor suppression protein, or any combinations thereof. In some embodiments, the antimicrobial peptide is magainin II, NRC-3, NRC-7, buforin IIb, BR2, or any combination thereof. In some embodiments, the cell penetrating peptide is Dox-TAT. In some embodiments, the tumor targeting peptide is RGD-SSL-Dox, LPD-PEG-NGR, or any combination thereof. In some embodiments, the therapeutic peptide targeting a transduction pathway is PNC-2, PNC-7, a cardiac natriuretic peptide, RGD-PEG-Suc-PD0325901, VWCS, FWCS, or any combination thereof. In some embodiments, the therapeutic peptide targeting a cell cycle is p16, Bac-7-ELP-p21, Pen-ELP-p21, or any combination therefor. In some embodiments, the therapeutic peptide inducing cell death is TAT-Bim, poropeptide-Bax, R8-Bax, CT20p-NP, RRM-MV, RRM-IL12, or any combination thereof. In some embodiments, the therapeutic peptide targeting a tumor suppression protein is PNC-27, PNC-21, PNC-28, or any combination thereof.

In some instances, the first anti-cancer therapy segment comprises a first promoter that controls the expression of a first nucleic acid sequence encoding an anti-cancer therapeutic. In some embodiments, the recombinant polynucleotide comprises a segment comprising a first nucleic acid sequence encoding (a) a first translation initiation signal and (b) an anti-cancer therapeutic, and a second anti-cancer therapy segment comprising a second nucleic acid sequence encoding (a) a second translation initiation signal and (b) an anti-cancer polypeptide, wherein said first translation initiation signal initiates the translation of the first nucleic acid sequence, and wherein said second translation initiation signal initiates the translation of the second nucleic acid sequence. In some embodiments, the second translation initiation signal is an internal ribosome entry site (IRES). In some embodiments, the method comprises introducing into the somatic cell or primary cell a recombinant polynucleotide described above, in which the recombinant polynucleotide gives rise, upon transcription, a factor that contributes to the reprogramming of the targeted somatic cell or targeted primary cell into an iTDC. In some embodiments, the factor is a transdifferentiation factor, e.g., Oct4, Sox2, Klf4, Myc, Ascl1, Brn2, Myt11, Olig2, or Zicl. In some embodiments, the factor is Sox2.

In some instances, the secretion of the anti-cancer therapeutic (e.g., TRAIL) is enhanced in the therapeutic cell when compared to an equivalent therapeutic cell which comprises a recombinant polynucleotide comprising the anti-cancer therapeutic, the transdifferentiation factor, and the anti-cancer polypeptide under a single translation initiation signal. In some embodiments, the secretion is enhanced by 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, or more.

In some embodiments, the secretion of TRAIL is enhanced in the therapeutic cell when compared to an equivalent therapeutic cell which comprises a recombinant polynucleotide comprising the TRAIL protein, the transdifferentiation factor, and the anti-cancer polypeptide under a single translation initiation signal. In some embodiments, the secretion is enhanced by 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, or more.

iii. Tumor Homing Mechanism

In some embodiments, the first and second therapeutic cells home to tumor cells. In some embodiments, the first and second therapeutic cells are induced tumor-homing drug carrier cells (iTDCs). In some embodiments, tumoritropic homing of a first and second iTDC is known to occur by chemotaxis. In some embodiments, the first and second iTDCs are able to follow a gradient of chemoattractants (e.g., pro-inflammatory cytokines and chemokines) released by tumor cells. In some embodiments, the chemoattractant is chemokine receptor type 4 (CXCR4). CXCR4 is an alpha chemokine receptor specific for stromal-derived-factor-1 (SDF-1). In some embodiments, chemoattractants (e.g., SDF-1) are released by the tumor cells into the blood stream and/or the cerebrospinal fluid (CSF). In some embodiments, following these signals, the first and second iTDCs rapidly reach the source of chemoattractants (i.e., the tumor cells) within and interact with the activated endothelial/ependymal cells near the tumor cells. In some embodiments, at this level, the first and second iTDCs and endothelial cells start an organized sequence of events resembling those described for T-cell extravasation that allow the selective entrance and specific homing of the transplanted first and second iTDCs in tumor areas. In some embodiments, the first and second iTDCs infiltrate the tumor. In some embodiments, migration of the first and second iTDCs towards tumor cells is dependent upon CXCR4-SDF-1 signaling.

iii. Matrix Compositions

In some embodiments, the treated surgical space is a tumor resection space. In some embodiments, an iTDC is administered to a tumor resection space via a matrix material. In some embodiments, the first iTDC is encapsulated in or attached to the matrix material. In some embodiments the first iTDC is attached to the scaffold prior to insertion of the matrix material into the surgical site. In some embodiments, the matrix material is flowable. In some embodiments, the matrix material comprises a hemostatic composition. In some embodiments, the matrix material is a hydrogel. In some embodiments, the matrix material comprises gelatin and/or thrombin. In some embodiments, the gelatin is bovine-derived gelatin. In some embodiments, the thrombin is human-derived. In some embodiments, the matrix material comprises fibrin.

In some embodiments, the matrix material comprises a component of extracellular matrix. In some embodiments, the component of extracellular matrix comprises a structural protein, a specialized protein, a glycosaminoglycan (GAG), a proteoglycan, or a combination thereof. In some embodiments, a structural protein comprises collagen, elastin, and fibrillin. In some embodiments, the collagen comprises collagen type I, collagen type IL collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, collagen type XXIX or a combination thereof. In some embodiments, the specialized protein comprises fibronectin, laminin, fibrinogen, tenascin, thrombospondin, integrin, or a combination thereof. In some embodiments, the glycosaminoglycan comprises a repeating disaccharide unit. In some embodiments, the disaccharide unit comprises a modified sugar and hexuronic acid. In some embodiments, the modified sugar comprises N-acetylgalactosamine (GalNAc), N-acetylglucosamine (GlcNAc), or a combination thereof. In some embodiments, the hexuronic acid comprises glucuronate (GlcA) or iduronate (IdA). In some embodiments, the glycosaminoglycan comprises hyaluronic acid, dermatan sulfate, chondroitin sulfate, heparin, heparin sulfate, and keratin sulfate. In some embodiments, the glycosaminoglycan is linked to core proteins, forming a proteoglycan. In some embodiments, the core proteins are rich in serine (Ser) and threonine (Thr) residues. In some embodiments, the proteoglycan further comprises a tetrasaccharide linker comprising a glucuronic acid (GlcA) residue, two galactose (Gal) residues, and a xylose (Xyl) residue. In some embodiments, the extracellular matrix is derived from a human, a cow, a horse, a sheep, a goat, a chimpanzee, a monkey, a rat, a pig, a mouse, a rabbit, or a synthetic reaction.

In some embodiments, the matrix material comprises a synthetic polymer, a natural polymer, or a combination thereof. In some embodiments, the matrix material is a gel. In some embodiments, the gel is a biogel or a hydrogel. In some embodiments, the synthetic polymer is polylactide (PLA), polycaprolactone (PCL), polyethylene glycol (PEG), a PEG macromer, polyethylene glycol methacrylate (PEGMA), polyethylene dimethacrylate (PEGDMA), poly(hydroxyethyl methacrylate) (PHEMA), polyvinyl alcohol (PVA), polyvinylpyrrolidone (PVP), carboxymethyl cellulose (CMC), polyimide (PI), polyacrylate (PAA), polyurethane (PU), PEG-lactide, PEG-glycolide or a combination thereof. In some embodiments, the gel does not comprise a synthetic polymer. In some embodiments, PEG macromers comprise reactive chain ends such as acrylate, methacrylate, allyl ether, maleimide, vinyl sulfone, NHS ester and vinyl ether groups. In some embodiments, the alcohol chain ends of PEG are esterified using acid chlorides (e.g., acryloyl chloride, methacryloyl chloride) in the presence of base. In some embodiments, PEG chain ends are etherified under basic conditions by reaction with alkyl halides such as 2-chloroethyl vinyl ether or allyl bromide. In some embodiments, acrylate, methacrylate, vinyl sulfone, maleimide, vinyl ether and allyl ether are capable of step growth network formation or polymerization. In some embodiments, polymerization of macromers is initiated using redox-generated radicals (e.g., ammonium persulfate and TEMED), or radicals generated with light. In some embodiments, the natural polymer is alginate, cellulose, gelatin, pectin, chitosan, paraffin, agarose, or a combination thereof. In some embodiments, the matrix material comprises Matrigel®. In some embodiments, the synthetic polymer or the natural polymer comprises a modification to enable crosslinking. In some embodiments, the modification to enable crosslinking is methacrylation. In some embodiments, the synthetic polymer or the natural polymer comprises a functional molecule. In some embodiments, the functional molecule comprises a bioactive protein or drug. In some embodiments, the synthetic polymer or the natural polymer comprises a peptide to promote cell adhesion, a peptide to promote proliferation, or a peptide to promote differentiation. In some embodiments, the peptide to promote cell adhesion is arginyl-glycyl-aspartic acid (RGD). In some embodiments, the synthetic polymer or the natural polymer comprises a biodegradable link. In some embodiments, the biodegradable link is a matrix metalloproteinase (MMP)-sensitive link or an Aggrecanase-sensitive link.

iv. Administration Schedules

In some embodiments, the second administration is performed simultaneously as the first administration. In some embodiments, the second administration is performed on the same day as the first administration. In some embodiments, the second administration is performed at most about 10 days after performing the first administration. In some embodiments, the second administration is performed at most about 6, 5, 4, 3, 2, or 1 day after the first administration.

In some embodiments, the second administration is performed about 1 day to about 20 days after performing the first administration. In some embodiments, the second administration is performed about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 15 days, about 1 day to about 20 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 15 days, about 2 days to about 20 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 15 days, about 3 days to about 20 days, about 4 days to about 5 days, about 4 days to about 6 days, about 4 days to about 7 days, about 4 days to about 8 days, about 4 days to about 9 days, about 4 days to about 10 days, about 4 days to about 15 days, about 4 days to about 20 days, about 5 days to about 6 days, about 5 days to about 7 days, about 5 days to about 8 days, about 5 days to about 9 days, about 5 days to about 10 days, about 5 days to about 15 days, about 5 days to about 20 days, about 6 days to about 7 days, about 6 days to about 8 days, about 6 days to about 9 days, about 6 days to about 10 days, about 6 days to about 15 days, about 6 days to about 20 days, about 7 days to about 8 days, about 7 days to about 9 days, about 7 days to about 10 days, about 7 days to about 15 days, about 7 days to about 20 days, about 8 days to about 9 days, about 8 days to about 10 days, about 8 days to about 15 days, about 8 days to about 20 days, about 9 days to about 10 days, about 9 days to about 15 days, about 9 days to about 20 days, about 10 days to about 15 days, about 10 days to about 20 days, or about 15 days to about 20 days after performing the first administration. In some embodiments, the second administration is performed about 1 day, about 2 days, about 3 days, about 4 days, about 5 days, about 6 days, about 7 days, about 8 days, about 9 days, about 10 days, about 15 days, or about 20 days. In some embodiments, the second administration is performed at least about 1 day, about 2 days, about 3 days, about 4 days, about 5 days, about 6 days, about 7 days, about 8 days, about 9 days, about 10 days, or about 15 days after performing the first administration. In some embodiments, the second administration is performed at most about 2 days, about 3 days, about 4 days, about 5 days, about 6 days, about 7 days, about 8 days, about 9 days, about 10 days, about 15 days, or about 20 days after performing the first administration.

v. Persistence

In some embodiments, the first iTDC persists in the subject for at least about 3 months after the first administration. In some embodiments, the first iTDC persists in the subject for about 7 days to about 180 days after the first administration. In some embodiments, the first iTDC persists in the subject for about 7 days to about 15 days, about 7 days to about 30 days, about 7 days to about 45 days, about 7 days to about 60 days, about 7 days to about 65 days, about 7 days to about 70 days, about 7 days to about 75 days, about 7 days to about 80 days, about 7 days to about 85 days, about 7 days to about 90 days, about 7 days to about 180 days, about 15 days to about 30 days, about 15 days to about 45 days, about 15 days to about 60 days, about 15 days to about 65 days, about 15 days to about 70 days, about 15 days to about 75 days, about 15 days to about 80 days, about 15 days to about 85 days, about 15 days to about 90 days, about 15 days to about 180 days, about 30 days to about 45 days, about 30 days to about 60 days, about 30 days to about 65 days, about 30 days to about 70 days, about 30 days to about 75 days, about 30 days to about 80 days, about 30 days to about 85 days, about 30 days to about 90 days, about 30 days to about 180 days, about 45 days to about 60 days, about 45 days to about 65 days, about 45 days to about 70 days, about 45 days to about 75 days, about 45 days to about 80 days, about 45 days to about 85 days, about 45 days to about 90 days, about 45 days to about 180 days, about 60 days to about 65 days, about 60 days to about 70 days, about 60 days to about 75 days, about 60 days to about 80 days, about 60 days to about 85 days, about 60 days to about 90 days, about 60 days to about 180 days, about 65 days to about 70 days, about 65 days to about 75 days, about 65 days to about 80 days, about 65 days to about 85 days, about 65 days to about 90 days, about 65 days to about 180 days, about 70 days to about 75 days, about 70 days to about 80 days, about 70 days to about 85 days, about 70 days to about 90 days, about 70 days to about 180 days, about 75 days to about 80 days, about 75 days to about 85 days, about 75 days to about 90 days, about 75 days to about 180 days, about 80 days to about 85 days, about 80 days to about 90 days, about 80 days to about 180 days, about 85 days to about 90 days, about 85 days to about 180 days, or about 90 days to about 180 days after the first administration. In some embodiments, the first iTDC persists in the subject for about 7 days, about 15 days, about 30 days, about 45 days, about 60 days, about 65 days, about 70 days, about 75 days, about 80 days, about 85 days, about 90 days, or about 180 days after the first administration. In some embodiments, the first iTDC persists in the subject for at least about 7 days, about 15 days, about 30 days, about 45 days, about 60 days, about 65 days, about 70 days, about 75 days, about 80 days, about 85 days, or about 90 days after the first administration. In some embodiments, the first iTDC persists in the subject for at most about 15 days, about 30 days, about 45 days, about 60 days, about 65 days, about 70 days, about 75 days, about 80 days, about 85 days, about 90 days, or about 180 days after the first administration.

In some embodiments, the second iTDC persists in the subject for at least about 12 hours after the second administration. In some embodiments, the second iTDC persists in the subject for about 1 hour to about 336 hours after the second administration. In some embodiments, the second iTDC persists in the subject for about 1 hour to about 4 hours, about 1 hour to about 8 hours, about 1 hour to about 12 hours, about 1 hour to about 24 hours, about 1 hour to about 48 hours, about 1 hour to about 72 hours, about 1 hour to about 96 hours, about 1 hour to about 168 hours, about 1 hour to about 336 hours, about 4 hours to about 8 hours, about 4 hours to about 12 hours, about 4 hours to about 24 hours, about 4 hours to about 48 hours, about 4 hours to about 72 hours, about 4 hours to about 96 hours, about 4 hours to about 168 hours, about 4 hours to about 336 hours, about 8 hours to about 12 hours, about 8 hours to about 24 hours, about 8 hours to about 48 hours, about 8 hours to about 72 hours, about 8 hours to about 96 hours, about 8 hours to about 168 hours, about 8 hours to about 336 hours, about 12 hours to about 24 hours, about 12 hours to about 48 hours, about 12 hours to about 72 hours, about 12 hours to about 96 hours, about 12 hours to about 168 hours, about 12 hours to about 336 hours, about 24 hours to about 48 hours, about 24 hours to about 72 hours, about 24 hours to about 96 hours, about 24 hours to about 168 hours, about 24 hours to about 336 hours, about 48 hours to about 72 hours, about 48 hours to about 96 hours, about 48 hours to about 168 hours, about 48 hours to about 336 hours, about 72 hours to about 96 hours, about 72 hours to about 168 hours, about 72 hours to about 336 hours, about 96 hours to about 168 hours, about 96 hours to about 336 hours, or about 168 hours to about 336 hours after the second administration. In some embodiments, the second iTDC persists in the subject for about 1 hour, about 4 hours, about 8 hours, about 12 hours, about 24 hours, about 48 hours, about 72 hours, about 96 hours, about 168 hours, or about 336 hours after the second administration. In some embodiments, the second iTDC persists in the subject for at least about 1 hour, about 4 hours, about 8 hours, about 12 hours, about 24 hours, about 48 hours, about 72 hours, about 96 hours, or about 168 hours. In some embodiments, the second iTDC persists in the subject for at most about 4 hours, about 8 hours, about 12 hours, about 24 hours, about 48 hours, about 72 hours, about 96 hours, about 168 hours, or about 336 hours after the second administration.

In some embodiments, the first and second iTDCs have rapid clearance through the lungs. In some embodiments, the first and second iTDCs clear the lungs in less than about 24 hours. In some embodiments, the first and second iTDCs have a half-life greater than at least 24 hours. In some embodiments, the first and second iTDCs have a half-life of about 48 hours. In some embodiments, the first and second iTDCs have a half-life of about 96 hours. In some embodiments, the first and second iTDCs have a half-life of about 120 hours. In some embodiments, the first and second iTDCs have a half-life of about 5 days. In some embodiments, the first and second iTDCs have a half-life of about 7 days.

B. Cancers

Provided herein are methods comprising: (a) performing a first administration, the first administration comprising administering a first induced tumor-homing drug carrier cell (iTDC) to a surgical space of a subject with cancer, the first iTDC comprising (i) a first recombinant nucleotide encoding a nucleic acid sequence encoding Sox2 and (ii) a first therapeutic nucleic acid sequence encoding a therapeutic payload; and (b) performing a second administration simultaneously, on the same day as the first administration, or at most about 7 days after performing the first administration, the second administration comprising parenterally administering a second iTDC to the subject, the second iTDC comprising (i) a second recombinant polynucleotide comprising a nucleic acid sequence encoding Sox2 and (ii) a second therapeutic nucleic acid sequence encoding a therapeutic payload; whereby the first and/or second iTDC expresses the therapeutic payload at a cancer cell and treats the cancer. In some instances, the cancer is a solid tumor. In other instances, the cancer is a hematologic malignancy. In additional instances, the cancer is a metastatic, relapsed, or refractory cancer.

i. Ovarian Cancer

In some embodiments, ovarian cancer is further classified into epithelial ovarian carcinoma, primary peritoneal carcinoma, and fallopian tube cancer. Epithelial ovarian carcinoma is the most common type of ovarian cancer, comprising about 85% to 90% of all cases. In some instances, epithelial ovarian carcinoma is further subtyped into mucinous, endometrioid, clear cell, and undifferentiated. In some embodiments, there are four stages of ovarian cancer. Stage I is characterized by cancerous cells localized within the ovary or fallopian tube (termed Stage IA), cancer that has developed in both ovaries or fallopian tubes but not on their outer surfaces (termed Stage IB), or cancer present in one or both ovaries or fallopian tubes in conjunction with any one of the following: 1) the tissue capsule surrounding the tumor broke during surgery, 2) cancer is on the outer surface of at least one of the ovaries, fallopian tubes, or tissue capsule, 3) cancerous cells were detected in fluid or washings from the abdomen (termed Stage IC). Stage II is characterized by the presence of cancerous cells in one or both ovaries or fallopian tubes that has invaded the uterus (termed Stage IIA) or cancerous cells that have grown into other nearby pelvic organs (termed Stage IIB). Stage III is characterized by the presence of cancerous cells in one or both ovaries or fallopian tubes, and in retroperitoneal lymph nodes (termed Stage IIIA1), or cancerous cells in one or both ovaries or fallopian tubes, in retroperitoneal lymph nodes, and in the lining of the upper abdomen (termed Stage IIIA2), or cancerous cells in one or both ovaries or fallopian tubes and cancer deposits 2 cm or smaller are in the abdomen (termed Stage IIIB), or cancerous cells in one or both ovaries or fallopian tubes and cancer deposits larger than 2 cm are in the abdomen (termed Stage IIIC). Stage IV is characterized by cancerous cells that have spread to the fluid around the lungs, with no other areas of cancer spread outside the pelvis or peritoneal cavity (termed Stage IVA) or cancerous cells that have spread to the inside of the spleen or liver, lymph nodes, and/or other organs or tissues outside the peritoneal cavity (termed Stage IVB).

In some embodiments, described herein is a cell therapy comprising administering a first iTDC into a surgical space of a subject with cancer and administering a second iTDC to the same subject, for the treatment of ovarian cancer. In some embodiments, described herein is a combination therapy which comprises administering to an individual in need thereof ganciclovir and a first and second iTDC, for the treatment of ovarian cancer. In some embodiments, described herein is a combination therapy which comprises administering to an individual in need thereof ganciclovir, a first and second iTDC, and chemotherapy, the treatment of ovarian cancer. In some embodiments, described herein is a combination therapy which comprises administering to an individual in need thereof ganciclovir, a first and second iTDC, and radiation therapy, for the treatment of ovarian cancer. In some embodiments, the ovarian cancer is epithelial ovarian carcinoma, primary peritoneal carcinoma, or fallopian tube cancer. In some embodiments, the ovarian cancer is a metastasized ovarian cancer. In some embodiments, the ovarian cancer is a relapsed or refractory ovarian cancer. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

ii. Glioblastoma

In some embodiments, the cancer is a brain cancer. In some embodiments, the brain cancer is a primary brain cancer. In some embodiments, the brain cancer is a secondary brain cancer. In some instances, the solid tumor is glioblastoma. Glioblastomas, or glioblastoma multiforme (GBM), are tumors that arise from astrocytes or the star-shaped cells that make up the “glue-like,” or supportive tissue of the brain. Glioblastoma is fast-growing, and in some embodiments, it is the most common primary tumor of the central nervous system in adults. In some embodiments, glioblastoma is further classified into primary glioblastoma (or de novo glioblastoma) or secondary tumor. In additional cases, glioblastoma is divided into grade I, grade II, grade III and grade IV glioblastoma.

In some embodiments, described herein is a cell therapy comprising administering a first iTDC into a surgical space of a subject with cancer and administering a second iTDC to the same subject, for the treatment of a tumor of the central nervous system (CNS). In some embodiments, described herein is a combination therapy which comprises administering to an individual in need thereof ganciclovir and a first and second iTDC, for the treatment of a tumor of the CNS. In some embodiments, described herein is a combination therapy which comprises administering to an individual in need thereof ganciclovir, a first and second iTDC, and chemotherapy, the treatment of a tumor of the CNS. In some embodiments, described herein is a combination therapy which comprises administering to an individual in need thereof ganciclovir, a first and second iTDC, and radiation therapy, for the treatment of a tumor of the CNS. In some embodiments, the tumor of the CNS is acoustic neuroma, grade I pilocytic astrocytoma, grade II low grade astrocytoma, grade III anaplastic astrocytoma, grade IV glioblastoma, chordoma, CNS lymphoma, craniopharyngioma, brain stem glioma, ependymoma, mixed glioma, optic nerve glioma, subependymoma, medulloblastoma, meningioma, oligodendrimoma, pituitary tumor, primitive neuroectodermal, Schwannoma, or any combination thereof. In some embodiments, the CNS tumor is a metastasized CNS tumor. In some embodiments, the CNS tumor is a relapsed or refractory CNS tumor. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

ii. Breast Cancer

Each year, there are about 2,300 new cases of breast cancer in men and about 230,000 new cases in women. In some embodiments, breast cancer is further classified into invasive ductal carcinoma (IDC), invasive lobular carcinoma (ILC), ductal carcinoma in situ, lobular carcinoma in situ, infiltrating ductal carcinoma, inflammatory breast cancer, triple-negative breast cancer, Paget disease of the nipple, phyllodes tumor, angiosarcoma, adenoid cystic carcinoma, adenoid cystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma and mixed carcinoma. IDC is the most common type of breast cancer, comprising about 80% of all cases. In some embodiments, there are four stages of breast cancer. In some instances, stage I is characterized by a tumor measuring up to 2 centimeters (cm) localized solely in the breast (termed Stage IA), small groups of cancerous cells found in the lymph nodes, or a tumor in the breast measuring up to 2 cm in addition to small groups of cancerous cells found in the lymph nodes (termed Stage IB). In some embodiments, stage II is characterized by the presence of a tumor larger than 2 millimeters (mm) localized in the lymph nodes (termed Stage IIA), by the presence of a tumor that is between 2 to 5 cm and small groups of cancerous cells localized in the lymph nodes, or a tumor that is larger than 5 cm but has not spread to the lymph nodes (termed Stage IIB). In some embodiments, stage III is characterized by the presence of a tumor larger than 5 cm localized in the lymph nodes (termed Stage IIIA), a tumor of any size that has spread to the chest wall and/or skin and has spread to up to 9 axillary lymph nodes or to the lymph nodes near the breastbone (termed Stage IIIB), or a tumor of any size that has spread to the chest wall and/or skin and has spread to 10 or more axillary lymph nodes or has spread to lymph nodes above or below the collarbone or has spread to axillary lymph nodes or to lymph nodes near the breast bone (termed Stage IIIC). In some embodiments, stage IV is characterized by cancerous cells that have spread beyond the breast and nearby lymph nodes to other organs of the body.

In some embodiments, described herein is a cell therapy comprising administering a first iTDC into a surgical space of a subject with cancer and administering a second iTDC to the same subject, for the treatment of breast cancer. In some embodiments, described herein is a combination therapy which comprises administering to an individual in need thereof ganciclovir and a first and second iTDC, for the treatment of breast cancer. In some embodiments, described herein is a combination therapy which comprises administering to an individual in need thereof ganciclovir, a first and second iTDC, and chemotherapy, the treatment of breast cancer. In some embodiments, described herein is a combination therapy which comprises administering to an individual in need thereof ganciclovir, a first and second iTDC, and radiation therapy, for the treatment of breast cancer. In some embodiments, the breast cancer is triple-negative breast cancer. In some embodiments, the breast cancer is invasive ductal carcinoma (IDC), invasive lobular carcinoma (ILC), ductal carcinoma in situ, lobular carcinoma in situ, infiltrating ductal carcinoma, inflammatory breast cancer, Paget disease of the nipple, phyllodes tumor, angiosarcoma, adenoid cystic carcinoma, adenoid cystic carcinoma, low-grade adenosquamous carcinoma, medullary carcinoma, mucinous carcinoma, colloid carcinoma, papillary carcinoma, tubular carcinoma, metaplastic carcinoma, spindle cell carcinoma, squamous carcinoma, micropapillary carcinoma, or mixed carcinoma. In some embodiments, the breast cancer is a metastasized breast cancer. In some embodiments, the breast cancer is a relapsed or refractory breast cancer. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

iii. Pulmonary Cancer

In some embodiments, the cancer is pulmonary cancer. In some embodiments, pulmonary cancer is further classified into non-small cell lung cancer (NSCLC), small cell lung cancer (SCLC), and lung carcinoid tumor. NSCLC is the most common type of pulmonary cancer, comprising about 85% of all cases. In some instances, NSCLC is further subtyped into adenocarcinoma, squamous cell carcinoma (or epidermoid carcinoma), and large cell carcinomas. In some embodiments, there are four stages of NSCLC. Stage I is characterized by cancerous cells localized in the lungs. Stage II is characterized by the presence of cancerous cells in the lung and nearby lymph nodes. Stage III is characterized by the presence of cancerous cells in the lung, and cancer either spreading to the lymph node on the same side of the chest as the original cancer (termed Stage IIIA), or cancer spreading to the lymph node on the opposite side of the chest as the original cancer (termed Stage IIIB). Stage IV is characterized by cancerous cells present in both lungs, in the pleural space surrounding the lungs, or in other parts of the body. In some embodiments, SCLC is subdivided into two stages, limited stage or extensive stage. Limited stage is characterized by the presence of cancerous cells on one side of the chest involving one part of the lung and nearby lymph nodes. Extensive stage is when cancer has spread to other regions of the chest or other parts of the body. Lung carcinoid tumor or lung neuroendocrine tumors, comprises the fewest cases, about less than 5% of all pulmonary cancer cases.

In some embodiments, described herein is a cell therapy comprising administering a first iTDC into a surgical space of a subject with cancer and administering a second iTDC to the same subject, for the treatment of pulmonary cancer. In some embodiments, described herein is a combination therapy which comprises administering to an individual in need thereof ganciclovir and a first and second iTDC, for the treatment of pulmonary cancer. In some embodiments, described herein is a combination therapy which comprises administering to an individual in need thereof ganciclovir, a first and second iTDC, and chemotherapy, the treatment of pulmonary cancer. In some embodiments, described herein is a combination therapy which comprises administering to an individual in need thereof ganciclovir, a first and second iTDC, and radiation therapy, for the treatment of pulmonary cancer. In some embodiments, the pulmonary cancer is non-small cell lung cancer (NSCLC), small cell lung cancer (SCLC), or lung carcinoid tumor. In some embodiments, the pulmonary cancer is a metastasized lung cancer. In some embodiments, the pulmonary cancer is a relapsed or refractory pulmonary cancer. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

iv. Melanoma

Melanoma is a type of cancer that is developed from melanocytes or pigment-containing cells. In some instances, melanoma occurs in the skin, but potentially also occurs in the mouth, intestines, or eye. Exemplary melanoma includes, but is not limited to lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a Spitz nevus, and uveal melanoma.

In some embodiments, described herein is a cell therapy comprising administering a first iTDC into a surgical space of a subject with cancer and administering a second iTDC to the same subject, for the treatment of melanoma. In some embodiments, described herein is a combination therapy which comprises administering to an individual in need thereof ganciclovir and a first and second iTDC, for the treatment of melanoma. In some embodiments, described herein is a combination therapy which comprises administering to an individual in need thereof ganciclovir, a first and second iTDC, and chemotherapy, the treatment of melanoma. In some embodiments, described herein is a combination therapy which comprises administering to an individual in need thereof ganciclovir, a first and second iTDC, and radiation therapy, for the treatment of melanoma. In some embodiments, the melanoma is lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a Spitz nevus, or uveal melanoma. In some embodiments, the melanoma is a metastasized melanoma. In some embodiments, the melanoma is a relapsed or refractory melanoma. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

v. Pancreatic Cancer

In some instances, the solid tumor is pancreatic cancer. Pancreatic cancer comprises two types, exocrine cancers such as pancreatic adenocarcinoma, acinar cell carcinoma of the pancreas, cystadenocarcinomas, pancreatoblastoma, adenosquamous carcinomas, signet ring cell carcinomas, hepatoid carcinomas, colloid carcinomas, undifferentiated carcinomas, undifferentiated carcinomas with osteoclast-like giant cells, or solid pseudopapillary tumor; and neuroendocrine malignant tumors.

In some embodiments, described herein is a cell therapy comprising administering a first iTDC into a surgical space of a subject with cancer and administering a second iTDC to the same subject, for the treatment of pancreatic cancer. In some embodiments, described herein is a combination therapy which comprises administering to an individual in need thereof ganciclovir and a first and second iTDC, for the treatment of pancreatic cancer. In some embodiments, described herein is a combination therapy which comprises administering to an individual in need thereof ganciclovir, a first and second iTDC, and chemotherapy, the treatment of pancreatic cancer. In some embodiments, described herein is a combination therapy which comprises administering to an individual in need thereof ganciclovir, a first and second iTDC, and radiation therapy, for the treatment of pancreatic cancer. In some embodiments, the pancreatic cancer is an acinar adenocarcinoma, ductal adenocarcinoma, intraductal papillary mucinous neoplasm (IPMN), acinar cell carcinoma, adenosquamous carcinoma, colloid carcinoma, giant cell tumor, hepatoid carcinoma, mucinous cystic neoplasms, pancreatoblastoma, serous cystadenoma, signet ring cell carcinoma, solid and pseudopapillary tumors, squamous cell carcinoma, undifferentiated carcinoma, insulinoma, glucagonoma, gastrinoma, somatotastinoma, VIPomas, PPomas, mucinous cystadenocarcinoma, non-functional islet cell tumor, squamous cell carcinomas, signet ring cell carcinomas, undifferentiated carcinomas, undifferentiated carcinomas with giant cells, carcinoma of the ampulla of Vater, or any combination thereof. In some embodiments, the pancreatic cancer is a metastasized pancreatic cancer. In some embodiments, the pancreatic cancer is a relapsed or refractory pancreatic cancer. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

vi. Leukemia

In some instances, the first iTDC and the second iTDC are administered to a subject for the treatment of a hematologic malignancy. In some instances, the hematologic malignancy is leukemia. Leukemia is a cancer that originates from the bone marrow and/or blood. Exemplary leukemia includes chronic myeloid leukemia (CML) (also known as chronic myelogenous leukemia), acute myeloid leukemia (AML) (also known as acute myelogenous leukemia), acute lymphocytic leukemia (ALL) (also known as acute lymphoblastic leukemia), and chronic lymphocytic leukemia (CLL).

In some embodiments, described herein is a cell therapy comprising administering a first iTDC into a surgical space of a subject with cancer and administering a second iTDC to the same subject, for the treatment of leukemia. In some embodiments, described herein is a combination therapy which comprises administering to an individual in need thereof ganciclovir and a first and second iTDC, for the treatment of leukemia. In some embodiments, described herein is a combination therapy which comprises administering to an individual in need thereof ganciclovir, a first and second iTDC, and chemotherapy, the treatment of leukemia. In some embodiments, described herein is a combination therapy which comprises administering to an individual in need thereof ganciclovir, a first and second iTDC, and radiation therapy, for the treatment of leukemia. In some embodiments, the pancreatic cancer is chronic myeloid leukemia (CML) (also known as chronic myelogenous leukemia), acute myeloid leukemia (AML) (also known as acute myelogenous leukemia), acute lymphocytic leukemia (ALL) (also known as acute lymphoblastic leukemia), and chronic lymphocytic leukemia (CLL), or any combination thereof. In some embodiments, the leukemia is metastasized leukemia. In some embodiments, the leukemia is a relapsed or refractory leukemia. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

vii. Lymphoma

In some instances, the hematologic malignancy is a lymphoma (e.g., a Hodgkin's lymphoma or a non-Hodgkin's lymphoma). Lymphoma is a cancer that is commonly manifested in the lymph nodes, lymphoid tissue, or lymphoid organs.

In some embodiments, described herein is a cell therapy comprising administering a first iTDC into a surgical space of a subject with cancer and administering a second iTDC to the same subject, for the treatment of lymphoma. In some embodiments, described herein is a combination therapy which comprises administering to an individual in need thereof ganciclovir and a first and second iTDC, for the treatment of lymphoma. In some embodiments, described herein is a combination therapy which comprises administering to an individual in need thereof ganciclovir, a first and second iTDC, and chemotherapy, the treatment of lymphoma. In some embodiments, described herein is a combination therapy which comprises administering to an individual in need thereof ganciclovir, a first and second iTDC, and radiation therapy, for the treatment of lymphoma. In some embodiments, the lymphoma is a Hodgkin's lymphoma or a non-Hodgkin's lymphoma. In some embodiments, the lymphoma is metastasized lymphoma. In some embodiments, the lymphoma is a relapsed or refractory lymphoma. In some embodiments, the first iTDC comprises a first recombinant polynucleotide comprising Sox2. In some embodiments, the second iTDC comprises a second recombinant polynucleotide comprising Sox2. In some embodiments, the first iTDC and the second iTDC secrete TK. In some embodiments, the first iTDC and the second iTDC secrete TRAIL.

C. Pharmaceutical Compositions

Disclosed herein, in certain embodiments, are pharmaceutical compositions comprising an iTDC and a pharmaceutically acceptable excipient.

Pharmaceutical compositions are formulated in a conventional manner using one or more pharmaceutically acceptable inactive ingredients that facilitate processing of the active compounds into preparations that are used pharmaceutically. Pharmaceutically-acceptable excipients included in the pharmaceutical compositions will have different purposes depending, for example, on the type of iTDCs used and the mode of administration. Non-limiting examples of generally used pharmaceutically-acceptable excipients include, without limitation: saline, buffered saline, dextrose, water-for-injection, glycerol, ethanol, dextran (e.g., low molecular dextran such as Dextran 40), PlasmaLyte, human serum albumin (HSA), and combinations thereof, stabilizing agents, solubilizing agents and surfactants, buffers and preservatives (such as dimethylsulfoxide (DMSO)), tonicity agents, bulking agents, and lubricating agents. The formulations comprising populations of iTDCs are prepared and cultured in the absence of any non-human components, such as animal serum.

In some embodiments, the pharmaceutical compositions further comprise a cryoprotectant or a cryopreservative. In some embodiments, the cryoprotectant or the cryopreservative is selected from dimethylsulfoxide (DMSO), formamide, propylene glycol, ethylene glycol, glycerol, trehalose, 2-methyl-2,4-pentanediol, methanol, butanediol, or any combination thereof.

Pharmaceutical compositions disclosed herein are administered to a subject using modes and techniques known to the skilled artisan. In some embodiments, the pharmaceutical compositions are for use in the cancer treatment methods described herein. In some embodiments, the pharmaceutical composition is administered directly to (e.g., deposited on) the treated surgical space. In some embodiments, the treated surgical space is a tumor resection margin or tumor resected area. In some embodiments, the tumor resection space comprises a matrix material as described herein. In some embodiments the pharmaceutical composition is attached to or encapsulated in a matrix material prior to the administration of the matrix material to the surgical site.

In some embodiments, the pharmaceutical composition is administered systemically to a target organ. In some embodiments, administration of the pharmaceutical composition is an intracerebroventricular (ICV) injection. In some embodiments, the pharmaceutical composition is administered via an intrathecal injection. In some embodiments, the pharmaceutical composition is administered via an intravenous injection. In some embodiments, the pharmaceutical composition is administered via an intraperitoneal injection.

Exemplary delivery modes include, but are not limited to, intraperitoneal injection. Other modes include, without limitation, intravenous, intratumoral, intradermal, subcutaneous (S.C., s.q., sub-Q, Hypo), intramuscular (i.m.), intra-arterial, intramedullary, intracardiac, intra-articular (joint), intrasynovial (joint fluid area), intracranial, intraspinal, intrathecal (spinal fluids), intraduodenal, intramedullary, intraosseous, intrathecal, intravascular, intravitreal, and epidural. In some embodiments, any known device useful for parenteral (e.g., intraperitoneal) injection and/or infusion of the formulations is used to effect such administration.

Formulations for injection are presented in unit dosage form, e.g., in ampoules or in multi-dose containers, with an added preservative. In some embodiments, the pharmaceutical compositions disclosed herein take such forms as suspensions, solutions, or emulsions in oily or aqueous vehicles, and contain formulatory agents such as suspending, stabilizing and/or dispersing agents. In some embodiments, the pharmaceutical compositions are presented in unit-dose or multi-dose containers, for example sealed ampoules and vials, and are stored in powder form or in a freeze-dried (lyophilized) condition requiring only the addition of the sterile liquid carrier, for example, saline or sterile pyrogen-free water, immediately prior to use. In some embodiments, extemporaneous injection solutions and suspensions are prepared from sterile powders, granules and tablets of the kind previously described.

In some embodiments, pharmaceutical compositions for parenteral administration include aqueous and non-aqueous (oily) sterile injection solutions of the active compounds which contain antioxidants, buffers, bacteriostats, and solutes which render the formulation isotonic with the blood of the intended recipient; and aqueous and non-aqueous sterile suspensions which include suspending agents and thickening agents. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. In some embodiments, the suspension also contains suitable stabilizers or agents which increase the solubility of the compounds to allow for the preparation of highly concentrated solutions.

It should be understood that in addition to the ingredients particularly mentioned above, the compounds and compositions described herein include other agents conventional in the art having regard to the type of formulation in question.

D. Combination Therapies

Disclosed herein, in certain embodiments, are methods of treating a cancer, comprising: (a) performing a first administration, the first administration comprising administering a first induced tumor-homing drug carrier cell (iTDC) to a surgical space of a subject with cancer, the first iTDC comprising (i) a first recombinant nucleotide encoding a nucleic acid sequence encoding Sox2 and (ii) a first therapeutic nucleic acid sequence encoding a therapeutic payload; and (b) performing a second administration simultaneously, on the same day as the first administration, or at most about 7 days after performing the first administration, the second administration comprising parenterally administering a second iTDC to the subject, the second iTDC comprising (i) a second recombinant polynucleotide comprising a nucleic acid sequence encoding Sox2 and (ii) a second therapeutic nucleic acid sequence encoding a therapeutic payload; whereby the first and/or second iTDC expresses the therapeutic payload at a cancer cell and treats the cancer

In some embodiments, the methods described herein further comprise administering an additional therapy. In some embodiments, the additional therapy is chemotherapy, radiation therapy, bone marrow transplant, immunotherapy, hormone therapy, cryoablation, radiofrequency ablation, or any combination thereof.

In some embodiments, the additional therapy is a chemotherapy. In some embodiments, the iTDCs are administered prophylactically in combination with the chemotherapeutic agent in order to treat cancer and/or a tumor. In some embodiments, the iTDCs are administered in combination with the chemotherapeutic agent in order to treat ovarian cancer. In some embodiments, the chemotherapeutic agent is an alkylating agent, an anthracycline, a cytoskeletal disruptor, an epothilone, a histone deacetylase inhibitor, a topoisomerase I inhibitor, a topoisomerase II inhibitor, a kinase inhibitor, a nucleotide analog, a precursor analog, a peptide antibiotic, a platinum-based agent, a retinoid, or a vinca alkaloid. In some embodiments, chemotherapeutic agents include: actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, and vinorelbine.

In some embodiments, the radiation therapy is an external beam radiation therapy. In some embodiments, the radiation therapy is an internal beam radiation therapy (brachytherapy). In some embodiments, the radiation therapy is a photon therapy. In some embodiments, photons are used in several different types of radiation therapy. In some embodiments, the radiation therapy is an orthovoltage radiation therapy, conventional radiation therapy, 3D conformal radiation therapy, Intensity Modulated Radiation Therapy (IMRT), brachytherapy, volumetric modulated arc therapy (also known as RapidArc®), and stereotactic radiation therapy (also known as radiosurgery, GammaKnife®, CyberKnife®, and stereotactic body radiation therapy (SBRT)), image-guided radiotherapy (IGRT), radiosurgery, proton therapy, electron radiation, or any combination thereof. Photon beams are the same type of beam that are used in diagnostic X-ray machines, such as those used to take chest X-rays; however, in radiotherapy, much higher energy photon beams are used. Conventional radiation therapy, 3D conformal radiation therapy, and IMRT are generally all delivered by machines called linear accelerators, or “linacs” for short.

In some embodiments, the radiation therapy is a two-dimensional (2D) photon therapy; also known as “conventional” radiation therapy. When 2D or conventional radiation therapy is used, X-rays films are used to determine how best to position the radiation beams in order to adequately treat tumors. Typically, a machine called a fluoroscopic simulator is used to plan the radiation treatments. The bones seen on the X-ray are used as landmarks to determine where the tumor is and where to position the radiation beams around the patient in order to treat the tumor, but avoid normal organs.

In some embodiments, the radiation therapy is a three-dimensional (3D) conformal radiation therapy, a computer tomography (CT)-guided radiation therapy, or a PET-guided radiation therapy. CT-guided therapy allows the tumor and normal organs to be defined in three dimensions, as opposed to using the “flat” image of an X-ray. In this type of therapy, a CT or PET scan, often referred to as a “simulation,” is obtained of the person in the position that they are to be treated. The tumor is then outlined in three dimensions on the CT scan. Normal organs that are located near the tumor and need to be avoided are also outlined in 3D. Beams are then arranged to best avoid normal organs, while delivering an optimal dose of radiation to the tumor. Computer software is then used to calculate the amount of radiation the tumor and normal tissues receive in order to assure that all parts of the tumor are covered sufficiently, while healthy organs receive as little radiation dose as possible. The beams of radiation can then be adjusted based on these calculations to further optimize the dose to the tumor and minimize the dose to normal organs.

In some embodiments, the additional therapy is a first-line treatment. In some embodiments, exemplary therapies include, but are not limited to, anti-cancer antibodies (e.g., HERCEPTIN®), antimetabolites, alkylating agents, topoisomerase inhibitors, microtubule targeting agents, kinase inhibitors, protein synthesis inhibitors, somatostatin analogs, glucocorticoids, aromatose inhibitors, mTOR inhibitors, protein Kinase B (PKB) inhibitors, phosphatidylinositol, 3-Kinase (PI3K) Inhibitors, cyclin dependent kinase inhibitors, anti-TRAIL molecules, MEK inhibitors, and the like.

In some embodiments, the additional therapy comprises a HSV-TK substrate. Exemplary HSV-TK substrates include, but are not limited to, FHBG (9-[4-fluoro-3-(hydroxymethyl)butyl]guanine), FHPG (9-([3-fiuoro-1-hydroxy 2 propoxy]meihyl)guaaine), FGCV (fluoroganciclovir), FPCV (fluoropenciclovir), FIAU (1-(2′-deoxy-2′-fiuoro-1-P-D-arabinofuranosyl)-5-iodouracil), FEAU (fluoro-5-ethyl-1-beta-D-arabinofuranosyluracil), FMAU (fluoro-5-methyl-1-beta-D-arabinofuranosyluracil), FHOMP (6-((1-fluoro-3-hydroxypropan-2-yloxy)methyl)-5-methylpryrimidine-2,4(1H,3H)-dione), ganciclovir, valganciclovir, acyclovir, valacivlovir, penciclovir, radiolabeled pyrimidine with 4-hydroxy-3-(hydroxymethyl)butyl side chain at N-1 (HHG-5-FEP) or 5-(2-)hydroxyethyl)- and 5-(3-hydroxypropyl)-substituted pyrimidine derivatives bearing 2,3-dihydroxypropyl, acyclovir-, ganciclovir and penciclovir-like side chains.

In some embodiments, the additional therapy include, but are not limited to flourouracil (5-FU), capecitabine/XELODA, 5-Trifluoromethyl-2′-deoxyuridine, methotrexate sodium, raltitrexed/Tomudex®, pemetrexed/Alimta®, cytosine Arabinoside (Cytarabine, Ara-C)/Thioguanine, 6-mercaptopurine (Mercaptopurine, 6-MP), azathioprine/Azasan®, 6-thioguanine (6-TG)/Purinethol (TEVA), pentostatin/Nipent®, fludarabine phosphate/Fludara®, cladribine (2-CdA, 2-chlorodeoxyadenosine)/Leustatin, floxuridine (5-fluoro-2)/FUDR (Hospira, Inc.), ribonucleotide Reductase Inhibitor (RNR), cyclophosphamide/Cytoxan (BMS), neosar, ifosfamide/Mitoxana, thiotepa, BCNU-1,3-bis(2-chloroethyl)-1-nitosourea, 1,-(2-chloroethyl)-3-cyclohexyl-lnitrosourea, methyl CCNU, hexamethylmelamine, busulfan/Myleran, procarbazine HCL/Matulane, dacarbazine (DTIC), chlorambucil/Leukaran®, melphalan/Alkeran, cisplatin (Cisplatinum, CDDP)/Platinol, carboplatin/Paraplatin, oxaliplatin/Eloxitan, bendamustine, carmustine, chloromethine, dacarbazine (DTIC), fotemustine, lomustine, mannosulfan, nedaplatin, nimustine, prednimustine, ranimustine, satraplatin, semustine, streptozocin, temozolomide, treosulfan, triaziquone, triethylene melamine, thioTEPA, triplatin tetranitrate, trofosfamide, uramustine, doxorubicin HCL/Doxil, daunorubicin citrate/Daunoxome®, mitoxantrone HCL/Novantrone, actinomycin D, etoposide/Vepesid, topotecan HCL/Hycamtin, teniposide (VM-26), irinotecan HCL(CPT-11), Camptosar®, camptothecin, Belotecan, rubitecan, vincristine, vinblastine sulfate, vinorelbine tartrate, vindesine sulphate, paclitaxel/Taxol, docetaxel/Taxotere, nanoparticle paclitaxel, abraxane, ixabepilone, larotaxel, ortataxel, tesetaxel, vinfiunine, and the like.

In some embodiments the additional therapy comprise one or more drugs selected from the group consisting of carboplatin(e.g., PARAPLATIN®), Cisplatin (e.g., PLATINOL®, PLATINOL-AQ®), Cyclophosphamide (e.g., CYTOXAN®, NEOSAPv®), Docetaxel (e.g., TAXOTERE®), Doxorubicin (e.g., ADRIAMYCIN®), Erlotinib (e.g., TARCEVA®), Etoposide (e.g., VEPESID®), Fluorouracil (e.g., 5-FU®), Gemcitabine (e.g., GEMZAR®), imatinib mesylate (e.g., GLEEVEC®), Irinotecan (e.g., CAMPTOSAR®), Methotrexate (e.g., FOLEX®, MEXATE®, AMETHOPTERIN®), Paclitaxel (e.g., TAXOL®, ABRAXANE®), Sorafmib (e.g., NEXAVAR®), Sunitinib (e.g., SUTENT®), Topotecan (e.g., HYCAMTIN®), Vinblastine (e.g., VELBAN®), Vincristine (e.g., ONCOVIN®, VINCASAR PFS®). In some embodiments, the additional therapeutic agents comprise one or more drugs selected from the group consisting of retinoic acid, a retinoic acid derivative, doxorubicin, vinblastine, vincristine, cyclophosphamide, ifosfamide, cisplatin, 5-fluorouracil, a camptothecin derivative, interferon, tamoxifen, and taxol. In some instances, the additional therapeutic agent is selected from the group consisting of abraxane, doxorubicin, pamidronate disodium, anastrozole, exemestane, cyclophosphamide, epirubicin, toremifene, letrozole, trastuzumab, megestroltamoxifen, paclitaxel, docetaxel, capecitabine, goserelin acetate, zoledronic acid, vinblastine, etc.), an antisense molecule, an siRNA, and the like.

In some embodiments, the additional therapy comprises an immune checkpoint modulator. Exemplary checkpoint modulators include:

PD-L1 modulators such as Genentech's MPDL3280A (RG7446), Avelumab (Bavencio) from Merck/Pfizer, durvalumab (Imfinzi) from AstraZeneca, Anti-mouse PD-L1 antibody Clone 10F.9G2 (Cat # BE0101) from BioXcell, anti-PD-L1 monoclonal antibody MDX-1105 (BMS-936559), BMS-935559 and BMS-986192 from Bristol-Meyer's Squibb, MSB0010718C, mouse anti-PD-L1 Clone 29E.2A3, CX-072 from XytomX Therapeutics, FAZ053 from Novartis Pharmaceuticals, KN035 from 3D Medicine, LY3300054 from Eli Lilly, and AstraZeneca's MEDI4736;

PD-L2 modulators such as GlaxoSmithKline's AMP-224 (Amplimmune), and rHIgM12B7;

PD-1 modulators such as anti-mouse PD-1 antibody Clone J43 (Cat # BE0033-2) from BioXcell, anti-mouse PD-1 antibody Clone RMP1-14 (Cat # BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, sintilimab (IBI-308) from Eli Lilly/Innovent Biologics, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, PDR001 from Novartis Pharmaceuticals, PF-06801591 from Pfizer, REGN2810 from Regeneron Pharmaceuticals, and Pidilizumab (CT-011) from CureTech Ltd;

CTLA-4 modulators such as Bristol Meyers Squibb's anti-CTLA-4 antibody ipilimumab (also known as Yervoy®, MDX-010, BMS-734016 and MDX-101), anti-CTLA4 antibody clone 9H10 from Millipore, Pfizer's tremelimumab (CP-675,206, ticilimumab), AGEN 1884 from Agenus, and anti-CTLA4 antibody clone BNI3 from Abcam;

LAG3 modulators such as anti-Lag-3 antibody clone eBioC9B7W (C9B7W) from eBioscience, anti-Lag3 antibody LS-B2237 from LifeSpan Biosciences, IMP701 and LAG525 from Novartis Pharmaceuticals, IMP321 (ImmuFact) from Immutep, anti-Lag3 antibody BMS-986016, BMS-986016 from Bristol-Myers Squibb, REGN3767 from Regeneron Pharmaceuticals, and the LAG-3 chimeric antibody A9H12;

B7-H3 modulators such as MGA271;

MR modulators such as Lirilumab (IPH2101) from Bristol-Myers Squibb;

CD137 modulators such as urelumab (BMS-663513, Bristol-Myers Squibb), PF-05082566 (anti-4-IBB, PF-2566, Pfizer), or XmAb-5592 (Xencor);

PS modulators such as Bavituximab;

OX40 modulators such as BMS-986178 from Bristol-Myers Squibb, GSK3174998 from GlaxoSmithKline, INCAGN1949 from Agenus, MEDI0562 from MedImmune, PF-04518600 from Pfizer, or RG7888 from Genentech;

GITR modulators such as GWN323 from Novartis Pharmaceuticals, INCAGN1876 from Agenus, or TRX518 from Leap Therapeutics;

TIM3 modulators such as MBG453 from Novartis Pharmaceuticals, or TSR-042 from TESARO;

and modulators such as an antibody or fragments (e.g., a monoclonal antibody, a human, humanized, or chimeric antibody) thereof, RNAi molecules, or small molecules to CD52, CD30, CD20, CD33, CD27, ICOS, BTLA (CD272), CD160, 2B4, LAIR1, TIGHT, LIGHT, DR3, CD226, CD2, or SLAM.

In some embodiments, the additional therapy comprises a cytokine therapy. Exemplary cytokine drugs include interferon gamma 1-b (Actimmune) from Horizon Pharma; IL-2 based recombinant fusion protein (ALKS 4230) from Alkermes; ALT-801 or ALT-803 from Altor BioScience; AM0010 from ARMO Biosciences; APN301 from Apeiron Biologics; CDX-301/CDX-1401 from Celldex; cergutuzumab amunaleukin (RG7813) or RG7461 from Genentch; CYT-6091 from Cytimmune Sciences; DI-Leu16-IL2 from Alopexx Oncology; GEN-1 from Celsion; heterodimeric IL-15 from Admune Therpeutics; HL143 from HanAll Biopharma; IGN002 from ImmunGene; ImmunoPulse IL-12 from OncoSec Medical; IRX-2 from IRX Therapeutics; M9241 (NHS-IL12) from EMD Serono; MDNA55 from Medicenna Therapeutics; NGR-hTNF from MolMed; or rSIFN-co from Sichuan Huiyang Life Science.

In some embodiments, the additional therapy comprises an adoptive cell therapy. Exemplary adoptive cell therapies include AFP TCR, MAGE-A10 TCR, or NY-ESO-TCR from Adaptimmune; ACTR087/rituximab from Unum Therapeutics; anti-BCMA CAR-T cell therapy, anti-CD19 “armored” CAR-T cell therapy, JCAR014, JCAR018, JCAR020, JCAR023, JCAR024, or JTCR016 from Juno Therapeutics; JCAR017 from Celgene/Juno Therapeutics; anti-CD19 CAR-T cell therapy from Intrexon; anti-CD19 CAR-T cell therapy, axicabtagene ciloleucel, KITE-718, KITE-439, or NY-ESO-1 T-cell receptor therapy from Kite Pharma; anti-CEA CAR-T therapy from Sorrento Therapeutics; anti-PSMA CAR-T cell therapy from TNK Therapeutics/Sorrento Therapeutics; ATA520 from Atara Biotherapeutics; AU101 and AU105 from Aurora BioPharma; baltaleucel-T (CMD-003) from Cell Medica; bb2121 from bluebird bio; BPX-501, BPX-601, or BPX-701 from Bellicum Pharmaceuticals; BSK01 from Kiromic; IMCgp100 from Immunocore; JTX-2011 from Jounce Therapeutics; LN-144 or LN-145 from Lion Biotechnologies; MB-101 or MB-102 from Mustang Bio; NKR-2 from Celyad; PNK-007 from Celgene; tisagenlecleucel-T from Novartis Pharmaceuticals; or TT12 from Tessa Therapeutics.

In some embodiments, the additional therapy comprises a proteasome inhibitor. Exemplary proteasome inhibitors include bortezomib, carfilzomib, delanzomib, ixazomib, marizomib, oprozomib, or derivatives or analogs thereof.

In some embodiments, the additional therapy comprises an HDAC inhibitor. Exemplary HDAC inhibitors include ACY-1215 (Rocilinostat), Apicidin, CI-994 (Tacedinaline), CR-2408, entinostat (SNDX-275 or MS-275), ITF2357 (Gavinostat), KD5170, JNJ-26481585, LBH589 (Panobinostat), NVP-LAQ824 (Dacinostat), PXD101 (Belinostat), romidepsin, phenyl butyrate (S-HDAC-42, AR-42), RAS2410 (Resminostat), sodium butyrate, suberoylanilide bis-hydroxamic acid (SBHA), trichostatin-A (TSA), tubacin, valproic acid (VPA), or vorinostat (SAHA).

In some instances, the first iTDC and the second iTDC are administered to the subject prior to administering the additional therapy.

In other instances, the first iTDC and the second iTDC are administered to the subject in conjunction with the additional therapy.

In additional instances, the first iTDC and the second iTDC are administered to the subject after administering the additional therapy.

In further instances, the subject undergoes radiation treatment, and the first iTDC and the second iTDC are administered to the subject before, during, or after radiation treatment.

In some embodiments, the additional therapy is administered simultaneously or on the same day as the first administration, the second administration, and/or the GCV or valganciclovir administration. In some embodiments, the additional therapy is performed about 1 day to about 90 days after the first administration, the second administration, and/or the GCV or valganciclovir administration. In some embodiments, the additional therapy is performed about 1 day to about 2 days, about 1 day to about 3 days, about 1 day to about 4 days, about 1 day to about 5 days, about 1 day to about 7 days, about 1 day to about 14 days, about 1 day to about 28 days, about 1 day to about 42 days, about 1 day to about 56 days, about 1 day to about 90 days, about 2 days to about 3 days, about 2 days to about 4 days, about 2 days to about 5 days, about 2 days to about 7 days, about 2 days to about 14 days, about 2 days to about 28 days, about 2 days to about 42 days, about 2 days to about 56 days, about 2 days to about 90 days, about 3 days to about 4 days, about 3 days to about 5 days, about 3 days to about 7 days, about 3 days to about 14 days, about 3 days to about 28 days, about 3 days to about 42 days, about 3 days to about 56 days, about 3 days to about 90 days, about 4 days to about 5 days, about 4 days to about 7 days, about 4 days to about 14 days, about 4 days to about 28 days, about 4 days to about 42 days, about 4 days to about 56 days, about 4 days to about 90 days, about 5 days to about 7 days, about 5 days to about 14 days, about 5 days to about 28 days, about 5 days to about 42 days, about 5 days to about 56 days, about 5 days to about 90 days, about 7 days to about 14 days, about 7 days to about 28 days, about 7 days to about 42 days, about 7 days to about 56 days, about 7 days to about 90 days, about 14 days to about 28 days, about 14 days to about 42 days, about 14 days to about 56 days, about 14 days to about 90 days, about 28 days to about 42 days, about 28 days to about 56 days, about 28 days to about 90 days, about 42 days to about 56 days, about 42 days to about 90 days, or about 56 days to about 90 days after the first administration, the second administration, and/or the GCV or valganciclovir administration. In some embodiments, the additional therapy is performed about 1 day, about 2 days, about 3 days, about 4 days, about 5 days, about 7 days, about 14 days, about 28 days, about 42 days, about 56 days, or about 90 days. In some embodiments, the additional therapy is performed at least about 1 day, about 2 days, about 3 days, about 4 days, about 5 days, about 7 days, about 14 days, about 28 days, about 42 days, or about 56 days after the first administration, the second administration, and/or the GCV or valganciclovir administration. In some embodiments, the additional therapy is performed at most about 2 days, about 3 days, about 4 days, about 5 days, about 7 days, about 14 days, about 28 days, about 42 days, about 56 days, or about 90 days after the first administration, the second administration, and/or the GCV or valganciclovir administration.

E. Methods of Dosing and Treatment Regimens

In certain embodiments, the iTDCs and/or the combination therapies described herein are administered for prophylactic and/or therapeutic treatments of diseases. In some embodiments, the iTDCs described herein are administered for prophylactic and/or therapeutic treatments of diseases. In certain therapeutic applications, the iTDCs are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, the iTDCs described herein are administered to a patient susceptible to or otherwise at risk of a particular disease, disorder or condition. Such an amount is defined to be a “prophylactically effective amount or dose.” In this use, the precise amounts also depend on the state of health of the patient, the weight of the patient, and the like. When used in patients, effective amounts for this use will depend on the severity and course of the disease, disorder or condition, previous therapy, the health status of the patient, response of the patient to the drugs, and the judgment of the treating physician. In one aspect, prophylactic treatments include administering to an individual, who previously experienced at least one symptom of the disease being treated and is currently in remission, a pharmaceutical composition comprising an iTDC described herein, in order to prevent a return of the symptoms of the disease or condition.

In certain embodiments, an iTDC and an additional therapeutic agent described herein are administered at a dose lower than the dose at which either the iTDC or the additional therapeutic agent are normally administered as monotherapy agents. In certain embodiments, an iTDC and an additional therapeutic agent described herein are administered at a dose lower than the dose at which either the iTDC or the additional therapeutic agent are normally administered to demonstrate efficacy. In certain embodiments, an iTDC is administered at a dose lower than the dose at which it is normally administered as a monotherapy agent, when administered in combination with an additional therapeutic agent described herein. In certain embodiments, an iTDC is administered at a dose lower than the dose at which it is normally administered to demonstrate efficacy, when administered in combination with an additional therapeutic agent described herein. In certain embodiments, an additional therapeutic agent is administered at a dose lower than the dose at which it is normally administered as a monotherapy agent, when administered in combination with an iTDC. In certain embodiments, an additional therapeutic agent is administered at a dose lower than the dose at which it is normally administered to demonstrate efficacy, when administered in combination with an iTDC.

In certain embodiments, wherein the condition of the patient does not improve, upon the discretion of the doctor, the administration of the therapeutic cell compositions are administered chronically, that is, for an extended period of time, including throughout the duration of the life of the patient in order to ameliorate or otherwise control or limit the symptoms of the patient's disease or condition.

In certain embodiments wherein a status of a patient does improve, the dose of the pharmaceutical compositions being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the conditions of the patient has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the individual in need of treatment, but nevertheless is determined according to the particular circumstances surrounding the case, including, e.g., the specific agent being administered, the route of administration, the condition being treated, and the individual being treated.

In some embodiments, the pharmaceutical compositions comprising an iTDC are administered at a dosage in the range of about 10³ to about 10¹⁰ therapeutic cell per kg of body weight, including all integer values within those ranges. In one embodiment, the desired dose is conveniently presented in a single dose or in divided doses administered simultaneously or at appropriate intervals, for example as two, three, four or more sub-doses per day. In some embodiments, the desired dose is administered as a single dose or in divided doses within about 72 hours of each other. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual, the severity of the disease or condition being treated, and the judgment of the practitioner.

In some embodiments, the pharmaceutical compositions comprising an iTDC are administered at a dosage of about 10×10⁶ cells per kilogram (kg). In some embodiments, the pharmaceutical compositions comprising an iTDC are administered at a dosage of about 12.5×10⁶ cells/kg. In some embodiments, the pharmaceutical compositions comprising an iTDC are administered at a dosage of about 1,000 cells/kg to about 1,000,000,000 cells/kg. In some embodiments, the pharmaceutical compositions comprising an iTDC are administered at a dosage of at least about 1,000 cells/kg. In some embodiments, the pharmaceutical compositions comprising an iTDC are administered at a dosage of at most about 1,000,000,000 cells/kg. In some embodiments, the pharmaceutical compositions comprising an iTDC are administered at a dosage of about 1,000 cells/kg to about 10,000 cells/kg, about 1,000 cells/kg to about 100,000 cells/kg, about 1,000 cells/kg to about 1,000,000 cells/kg, about 1,000 cells/kg to about 10,000,000 cells/kg, about 1,000 cells/kg to about 100,000,000 cells/kg, about 1,000 cells/kg to about 1,000,000,000 cells/kg, about 10,000 cells/kg to about 100,000 cells/kg, about 10,000 cells/kg to about 1,000,000 cells/kg, about 10,000 cells/kg to about 10,000,000 cells/kg, about 10,000 cells/kg to about 100,000,000 cells/kg, about 10,000 cells/kg to about 1,000,000,000 cells/kg, about 100,000 cells/kg to about 1,000,000 cells/kg, about 100,000 cells/kg to about 10,000,000 cells/kg, about 100,000 cells/kg to about 100,000,000 cells/kg, about 100,000 cells/kg to about 1,000,000,000 cells/kg, about 1,000,000 cells/kg to about 10,000,000 cells/kg, about 1,000,000 cells/kg to about 100,000,000 cells/kg, about 1,000,000 cells/kg to about 1,000,000,000 cells/kg, about 10,000,000 cells/kg to about 100,000,000 cells/kg, about 10,000,000 cells/kg to about 1,000,000,000 cells/kg, about 100,000,000 cells/kg to about 1,000,000,000 cells/kg. In some embodiments, the pharmaceutical compositions comprising an iTDC are administered at a dosage of about 1,000 cells/kg, about 10,000 cells/kg, about 100,000 cells/kg, about 1,000,000 cells/kg, about 10,000,000 cells/kg, about 100,000,000 cells/kg, or about 1,000,000,000 cells/kg.

In certain instances, it is appropriate to administer at least one pharmaceutical composition described herein, in combination with one or more other therapeutic agents.

In one embodiment, the therapeutic effectiveness of one of the pharmaceutical compositions described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the pharmaceutical compositions described herein with another agent (which also includes a therapeutic regimen) that also has therapeutic benefit.

In one specific embodiment, a pharmaceutical composition described herein, is co-administered with a second therapeutic agent, wherein the pharmaceutical composition described herein, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is additive of the two therapeutic agents or the patient experiences a synergistic benefit.

In certain embodiments, different dosages of the pharmaceutical composition disclosed herein are utilized in formulating pharmaceutical composition and/or in treatment regimens when the compounds disclosed herein are administered in combination with one or more additional agent, such as an additional drug, an adjuvant, or the like. Dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a pharmaceutical composition described herein, is initiated prior to, during, or after treatment with a second agent described herein, and continues until any time during treatment with the second agent or after termination of treatment with the second agent. It also includes treatments in which a pharmaceutical composition described herein, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the individual suffers; the age, weight, sex, diet, and medical condition of the individual). Thus, in some instances, the dosage regimen actually employed varies and, in some embodiments, deviates from the dosage regimens set forth herein.

For combination therapies described herein, dosages of the co-administered pharmaceutical compositions vary depending on the type of co-drug employed, on the specific drug employed, on the disease or condition being treated and so forth. In additional embodiments, when co-administered with one or more other therapeutic agents, the pharmaceutical composition provided herein is administered either simultaneously with the one or more other therapeutic agents, or sequentially.

In combination therapies, the multiple therapeutic agents (one of which is one of the pharmaceutical compositions described herein) are administered in any order or even simultaneously. If administration is simultaneous, the multiple therapeutic agents are, by way of example only, provided in a single, unified form, or in multiple forms (e.g., as a single pill or as two separate pills).

The pharmaceutical compositions described herein, or a pharmaceutically acceptable salt thereof, as well as combination therapies, are administered before, during or after the occurrence of a disease or condition, and the timing of administering the pharmaceutical composition containing a compound varies. Thus, in one embodiment, the pharmaceutical compositions described herein are used as a prophylactic and are administered continuously to individuals with a propensity to develop conditions or diseases in order to prevent the occurrence of the disease or condition. In another embodiment, the pharmaceutical compositions are administered to an individual during or as soon as possible after the onset of the symptoms. In specific embodiments, a pharmaceutical composition described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each individual. For example, in specific embodiments, a compound described herein or a formulation containing the pharmaceutical composition is administered for at least 2 weeks, about 1 month to about 5 years.

F. Methods of Target Site Visualization

Described herein, in certain embodiments, are methods of imaging an iTDC at a target site of interest. In some embodiments, the methods comprise: (a) performing a first administration, the first administration comprising administering a first induced tumor-homing drug carrier cell (iTDC) to a surgical space of a subject with cancer, the first iTDC comprising a first recombinant nucleotide encoding a nucleic acid sequence encoding Sox2; (b) performing a second administration simultaneously, on the same day as the first administration, or at most about 7 days after performing the first administration, the second administration comprising parenterally administering a second iTDC to the subject, the second iTDC comprising a second recombinant polynucleotide comprising a nucleic acid sequence encoding Sox2; and (c) imaging the target site of interest. In some embodiments, the cancer is a solid cancer. In some embodiments, the cancer is a lung cancer, breast cancer (e.g., triple negative breast cancer), brain cancer (e.g., glioblastoma), ovarian cancer, pulmonary cancer, melanoma, leukemia, lymphoma, and pancreatic cancer.

In some embodiments, the methods of imaging an iTDC at a target site of interest comprise the use of an imaging modality. In some embodiments, the imaging modality is positron emission tomography (PET). In some embodiments, the imaging modality is magnetic resonance imaging (MRI), ultrasound, X-ray imaging, computer tomography (CT), nuclear medicine, elastography, photoacoustic imaging, echocardiography, functional near-infrared spectroscopy, magnetic particle imaging, or any combination thereof. In some embodiments, the imaging modality uses a volume rendering technique to produce a three-dimensional (3D) image. In some embodiments, the imaging modality is fluorescence microscopy, confocal microscopy, bright field microscopy, oblique illumination microscopy, dark field microscopy, dispersion staining microscopy, phase contrast microscopy, differential interference contrast microscopy, electron microscopy, scanning probe microscopy, ultraviolet microscopy, infrared microscopy, digital holographic microscopy, digital pathology microscopy, laser microscopy, photoacoustic microscopy, or any combinations thereof.

In some embodiments, the first and second iTDCs comprise a label. In some embodiments, the imaging modality detects the label. In some embodiments, the label is a fluorescent label. In some embodiments, the first recombinant polynucleotide and the second recombinant polynucleotide encode a nucleic acid sequence encoding the fluorescent label. Non-limiting examples of fluorescent labels include green fluorescent protein, tdTomato, E2-Crimson, mCherry, red fluorescent protein, cyan fluorescent protein, or any combination thereof. In some embodiments, the label is a magnetic resonance imaging (MRI) contrast agent or a positron emission tomography (PET) contrast agent. In some embodiments, thymidine kinase is able to be detected via PET. Thymidine kinase (TK) is amenable to detection via PET.

In some embodiments, thymidine kinase is the label. In some embodiments, TK phosphorylates ganciclovir. In some embodiments, phosphorylated ganciclovir is the label. In some embodiments, phosphorylated TK accumulates inside cells, thereby enabling detection via PET. In some embodiments, the uptake of TK is regulated by thymidine kinase 1, and it is therefore taken up preferentially by rapidly proliferating tumor cells. The fluorine isotope 18 is a positron emitter that is used in positron emission tomography (PET). The fluorine-18 radiolabeled fluorothymidine F-18 is therefore useful for PET imaging of active tumor proliferation, and compares favorably with the more commonly used marker fludeoxyglucose (18F). In some embodiments, the label is fluorothymidine F-18.

In some embodiments, the target is site is a tumor. In some embodiments, the target site is a solid tumor. In some embodiments, the target is site is a cancer cell. In some embodiments, the target is site is an inflamed tissue or an area of inflammation in a tissue. In some embodiments, the target is site is an atherosclerotic plaque. In some embodiments, the target is site is a bone, cartilage, or tendon. In some embodiments, the target is site is a tumor resection area. In some embodiments, the target is site is a blood vessel.

In some embodiments, the methods disclosed herein comprise performing a first administration of the iTDCs to the subject in need thereof. In some embodiments, the first administration comprises administering a first iTDC to a surgical space of a subject with cancer. In some embodiments, the surgical space is a tumor resection space. In some embodiments, the tumor resection space is an area in or on the body of the subject comprising the tissue left after a resection (i.e., surgery to remove part or all of a tumor along with a margin of normal tissue surrounding the tumor) is performed. In some embodiments, the tumor resection space is the area of in or on the body of the subject left after a lung resection. In some embodiments, the tumor resection space is the area of in or on the body of the subject left after a mastectomy resection. In some embodiments, the tumor resection space is the area of in or on the body of the subject left after a brain tumor resection. In some embodiments, the tumor resection space is the area of in or on the body of the subject left after a pancreatic tumor resection. In some embodiments, the tumor resection space is the area of in or on the body of the subject left after an oophorectomy.

### II. Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Treatment of Glioblastoma Using the Therapeutic Cells of the Disclosure

A patient is diagnosed with glioblastoma multiforme. The patient undergoes a brain tumor resection to surgically remove the solid tumor. In order to halt the residual bleeding in the tumor resection area, the surgeon applies a hemostatic matrix to all bleeding areas within the tumor resection area. The physician administers a first iTDC directly onto and/or into the hemostatic matrix and continues to finish the surgery. The patient is administered: 1) an oral dose of ganciclovir, and 2) an intravenous dose of the second iTDC, upon regaining consciousness. The first iTDC and the second iTDC home to remnant glioblastoma cells and induce apoptosis. The first iTDC persists in the patient for about 3 months, and the second iTDC persists in the patient for about 1 week after the intravenous dose.

### Example 2: In Vivo Assessment of the Treatment of Primary Lung and Breast Cancer Using the Therapeutic Cells of the Disclosure

The therapeutic effect of the targeted cell therapy of the disclosure against primary non-small cell lung cancer (NSCLC) and primary triple-negative breast cancer was investigated.

House-bred nude mice were inoculated with fluorescence and bioluminescence labeled NSCLC SKMES lung tumor or MB231-Br fat-pad tumor by orthotopic implantation into the lung and subcutaneous implantation, respectively. After 7 days, therapeutic cells labeled with fluorescent and bioluminescent markers were administered by intravenous injection. In order to investigate the migration and persistence of the therapeutic cells, the tumors were harvested on day 3, day 7, and day 14 for sectioning. The therapeutic efficacy of the therapeutic cells was investigated by evaluating tumor volume by measuring the bioluminescence signal after administration of the therapeutic cells encoding TRAIL.

After cell systemic injection, the therapeutic cells were observed in the lung as early as Day 1, and they persisted in the lung for about 2 weeks. In comparison to the control group, the NSCLC tumor volume in the animals treated with the cytotoxic therapeutic cells was significantly smaller. The median survival of the treatment group was double of that for the control group (median survival 33 vs 67 days). For the triple-negative breast cancer model, the therapeutic cells migrated to the fat-pad tumor in as little as 3 days after cell administration. The number of cells that migrated to the tumor peaked at Day 7, and cells were still present in the tumor 2 weeks after injection. The average tumor volume in the animals treated with cytotoxic therapeutic cells was only 1/4 of that in the control group 14 days after therapy administration.

The therapeutic cells were able to migrate to a tumor site and reduce tumor burden after a single dose of systemic cell administration in both the NSCLC mouse model and the triple-negative primary breast cancer mouse model.

### Example 3: In Vitro and In Vivo Assessment of the Treatment of Brain Metastases Using the Therapeutic Cells of the Disclosure

The therapeutic cells of the disclosure were used as a targeted drug delivery system to scavenge for metastases in the brain. The therapeutic cells have demonstrated a remarkable, innate ability to selectively migrate to tumors. When engineered to produce cytotoxic proteins, the therapeutic cells have been proven to migrate to and kill glioblastoma tumors. The therapeutic cells were produced by rapidly transdifferentiating human fibroblasts into the therapeutic cells using a lentivirus containing the gene to induce expression of the multipotency transcription factor, Sox2. In some embodiments, the therapeutic cells are a personalized, tumor-homing therapeutic cell line.

The therapeutic cells were investigated in vitro and in vivo to treat cancer that has metastasized to the brain. The therapeutic cells were delivered via intracerebroventricular (ICV) injections to brain tumors in mice. ICV devices that allow for long-term, repeated dosing are well-established in the clinic. Additionally, ICV injections distribute cells throughout the brain, reducing the distance the migration distance to the tumor and allowing for the possibility of treating multiple tumor foci.

In order to assess the cells' therapeutic potential for cancer that has metastasized to the brain, treatment optimization was performed in vitro. Furthermore, in vivo studies were used to determine the migration, persistence, and efficacy of therapeutic the therapeutic cells against SKMES NSCLC and triple-negative breast cancer MB231-Br tumors in the brain. Following implantation of tumors into the brains of nude mice, the therapeutic cells were injected into the ventricle contralateral to the site of tumor implantation. The migration and persistence of the therapeutic cells was investigated by following the bioluminescent signal of the therapeutic cells as well as by sectioning following sacrifice. The therapeutic efficacy of the therapeutic cells was determined by following the bioluminescent signals. It was observed that the therapeutic cells induced significant tumor death in vitro; therefore, the migration and persistence of the therapeutic cells in vivo was investigated. Migration of the therapeutic cells to tumors in the hemisphere contralateral to the site of ICV injection of the therapeutic cells within 7 days was observed. Maximum therapeutic cell bioluminescent signal was observed 4 days after the therapeutic cell injection and persisted for more than one week. In both NSCLC and triple negative breast cancer models, the therapeutic cells reduced the growth rate of brain tumors by at least one order of magnitude.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### III. Definitions

Throughout this application, various embodiments of this disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “subject,” “individual,” “host,” “donor,” and “patient” are used interchangeably herein to refer to a vertebrate, for example, a mammal. Mammals include, but are not limited to, murine (e.g., mice and rats), simians, humans, farm animals (livestock and horses), sport animals, and pets (e.g., dogs and cats). Subjects may be of any age, including infant, juvenile, adolescent, adult, and geriatric subjects. Tissues, cells, and their progeny of a biological entity obtained in vivo or cultured in vitro are also encompassed. Designation as a “subject,” “individual,” “host,” “donor,” or “patient” does not necessarily entail supervision of a medical professional.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “therapeutically effective amount” refers to an amount of therapeutic cell or a pharmaceutical composition described herein that is sufficient and/or effective in achieving a desired therapeutic effect in treating a patient having disease or disorder. In some embodiments, a therapeutically effective amount of the therapeutic cell or composition will avoid adverse side effects.

As used herein, the terms “allogeneic” or “allogenic” means the therapeutic cells are obtained from a genetically non-identical donor. For example, allogenic iTDCs are extracted from a donor and returned back to a different, genetically non-identical recipient.

As used herein, the term “autologous” means the plurality of iTDCs are obtained from a genetically identical donor. For example, autologous iTDCs are extracted from a patient and returned back to the same, genetically identical patient.

As used herein, the terms “transdifferentiation” or “transdifferentiating” refer to a method in which differentiated somatic cells are directly converted to differentiated or multipotent somatic cells of a different lineage without passing through an intermediate pluripotent stem cell (iPSC) stage.

As used herein, the term “transdifferentiation factor” refers to a protein such as a transcription factor that promotes the direct conversion of one somatic cell type to another. Examples include, but are not limited to, Oct4, Sox2, Klf4, Myc, Ascl1, Brn2, Myt11, Olig2, Zicl, or any combinations thereof.

As used herein, the terms “treat” or “treatment” refer to any type of treatment that imparts a benefit to a patient afflicted with a disease or disorder. These terms include treatment directed toward removal of the cause of the disease or disorder, treatment directed to the relief of symptoms, treatment directed to inhibiting the development of the disease or disorder, and treatment that supplements another therapy directed towards the improvement of the disease or disorder. Non-limiting examples of the disease or disorder include a cancer, a neurodegenerative disorder, and a neural trauma. Non-limiting examples of the benefit imparted to a patient afflicted with a disease or disorder include improvement in the condition of the patient (e.g., in one or more symptoms), a delay in the progression of the disease or disorder, and a delay in an onset or a recurrence of the disease.

As used herein, the term “transfecting” is the transfer of heterologous genetic material into a cell, often through the use of a vector (e.g., molecule used as a vehicle to carry foreign genetic material into another cell). Methods of transfecting eukaryotic cells are known, and may include, but are not limited to, electroporation, use of cationic liposome-based reagents, nanoparticle-based reagents, polymeric-based reagents, polymeric and liposomal-based reagents, or any combination thereof.

As used herein, the term “transducing” is the transfer of heterologous genetic material into a cell by means of a virus. Such viral vectors are known and may include, but are not limited to, lentiviral vectors, adenoviral vectors, retroviral or any combination thereof.

1.-46. (canceled)

47. A method of treating a cancer in an individual in need thereof, comprising:
a. performing a first administration, the first administration comprising administering to a tumor resection space of the individual a composition comprising a first induced tumor-homing drug carrier cell (iTDC) and a matrix material, the first iTDC comprising (i) a nucleic acid sequence encoding Sox2, and (ii) a first therapeutic nucleic acid sequence encoding a first therapeutic payload; and
b. performing a second administration simultaneously, on the same day as the first administration, or at most about 7 days after performing the first administration, the second administration comprising parenterally administering a composition comprising a second iTDC to the individual, the second iTDC comprising (i) a nucleic acid sequence-encoding Sox2, and (ii) a second therapeutic nucleic acid sequence encoding a second therapeutic payload,
wherein the first and second iTDC each express the therapeutic payload at a cancer cell, thereby treating the cancer.

48. The method of claim 47, wherein the second administration is performed within about 24 hours of the first administration.

49. The method of claim 47, wherein the second administration is performed no more than 5 days after the first administration.

50.-51. (canceled)

52. The method of claim 47, wherein the matrix material is a hydrogel.

53. The method of claim 47, wherein the matrix material comprises gelatin, thrombin, fibrin, or a combination thereof.

54.-56. (canceled)

57. The method of claim 47, wherein the cancer is selected from brain cancer, ovarian cancer, pulmonary cancer, breast cancer, melanoma, pancreatic cancer, leukemia, and lymphoma.

58. The method of claim 57, wherein the brain cancer is glioblastoma (GBM).

59.-61. (canceled)

62. The method of claim 61, wherein the breast cancer is triple negative breast cancer.

63.-67. (canceled)

68. The method of claim 47, wherein the first and/or second therapeutic payload comprises thymidine kinase (TK).

69. (canceled)

70. The method of claim 47, wherein the first and/or second therapeutic payload comprises tumor necrosis factor (TNF), monocyte chemoattractant protein (MCP), IL-1-like cytokine, gamma chain cytokine, beta chain cytokine, IL-6-like cytokine, IL-10-like cytokine, interferon, tumor necrosis factor, TGF-beta, macrophage inflammatory protein (MIP), tumor growth factor (TGF), matrix metalloprotease (MMP), or any combination thereof.

71. (canceled)

72. The method of claim 47, wherein the first and/or second therapeutic payload comprises TNF-related apoptosis-inducing ligand (TRAIL), or secretable TRAIL (s-TRAIL).

73. The method of claim 68, further comprising administering ganciclovir (GCV) or valganciclovir to the individual within at least 3 days of the first administration and the second administration.

74.-76. (canceled)

77. The method of claim 71, further comprising administering to the individual a chemotherapeutic agent, radiation therapy, bone marrow transplant, immunotherapy, hormone therapy, cryoablation, radiofrequency ablation, or any combination thereof.

78. (canceled)

79. The method of claim 77, wherein the chemotherapeutic agent is an alkylating agent, an anthracycline, a cytoskeletal disruptor, an epothilone, a histone deacetylase inhibitor, a topoisomerase I inhibitor, a topoisomerase II inhibitor, a kinase inhibitor, a nucleotide analog, a precursor analog, a peptide antibiotic, a platinum-based agent, a retinoid, a vinca alkaloid, or a combination thereof.

80. The method of claim 77, wherein the chemotherapeutic agent is actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, or a combination thereof.

81.-82. (canceled)

83. The method of claim 47, wherein the first iTDC and the second iTDC are autologous cells to the individual.

84. The method of claim 47, wherein the second administration is an intracerebroventricular (ICV) injection, intrathecal injection, intravenous injection, or intraperitoneal injection.

85.-89. (canceled)

90. The method of claim 47, wherein the first iTDC and/or the second iTDC comprise a fluorescent label.

91.-93. (canceled)

94. The method of claim 47, wherein the first and/or second therapeutic payload comprises TK and TRAIL.

95. The method of claim 94, further comprising administering ganciclovir (GCV) or valganciclovir to the individual within at least 3 days of the first administration and the second administration.
